(19) United States
(12) Patent Application Publication
(10) Pub. No.: US 2016/0310151 A1
(43) Pub. Date: Oct. 27, 2016

(54) MEDICAL DEVICE AND MEDICAL DEVICE ASSEMBLY

(71) Applicant: TERUMO KABUSHIKI KAISHA, Tokyo (JP)

(72) Inventors: Kinya HARADA, Fuji-city (JP); Yuusuke SEKINE, Chigasaki-city (JP)

(73) Assignee: TERUMO KABUSHIKI KAISHA, Tokyo (JP)

(21) Appl. No.: 15/135,694

(22) Filed: Apr. 22, 2016

(30) Foreign Application Priority Data
Apr. 24, 2015 (JP) 2015-089724

(51) Int. Cl. A61B 17/22; A61B 17/3207

(52) U.S. Cl. CPC A61B 17/22; A61B 17/32075; A61B 17/320758; A61B 2017/22001; A61B 2017/22038; A61B 2017/22094; A61B 2017/00778

(57) ABSTRACT

The medical device includes a catheter that has a first lumen into which a medical wire is insertable. The medical device also has a tube-shaped body proximal to the catheter, and the tube-shaped body includes a second lumen which communicates with the first lumen. A delivery mechanism is accommodated in the second lumen of the tube-shaped body. The delivery mechanism enables a delivery operation for moving the medical wire distally beyond the distal end of the medical device by a predetermined movement amount.

### CROSS-REFERENCES TO RELATED APPLICATIONS

This application is based on and claims priority to Japanese Application No. 2015-089724 filed on Apr. 24, 2015, the entire content of which is incorporated herein by reference.

### TECHNICAL FIELD

The present invention relates to a medical device and a medical device assembly.

### BACKGROUND DISCUSSION

A catheter device such as a balloon catheter is generally used to treat a stenosed site in a biological lumen. In a technique using the catheter device, such as the micro catheter disclosed in Japanese Patent Application Publication No. JP-T-2007-516008, a guide wire is first inserted into the stenosed site. Thereafter, the catheter device is inserted into the stenosed site along the guide wire to perform treatment for expanding the stenosed site.

In some cases, depending on a progressed condition of symptoms of the stenosed site, the stenosed site is occluded to such an extent that the guide wire is less likely to be inserted (i.e., it is difficult to insert the guide wire into the stenosed site). In this case, for example, an operator presses the guide wire into the stenosed site by using the operator's hand outside a living body, or tries to insert a penetrating wire (i.e., a wire for penetrating the stenosed site) into the stenosed site by separately delivering the penetrating wire to the stenosed site via the micro catheter.

However, a medical wire such as the guide wire, the penetrating wire, and the like is generally configured to include a flexible and elongated member in order to ensure that the medical wire is able to be inserted into a curved and meandering biological lumen. Consequently, a user is less likely to concurrently perform the user's hand side operation and an operation in a distal portion of the medical wire. The user has some difficulties in finely adjusting a movement amount of the medical wire or in transmitting a sufficient pressing force to the distal portion of the medical wire by the user's hand side operation. Therefore, in some cases the user cannot efficiently perform the treatment for causing the medical wire to penetrate the stenosed site, thereby leading to a long delay in an operation time.

### SUMMARY

The medical device and the medical device assembly disclosed here are designed in view of the above-described problem. The disclosed medical device and the medical device assembly can easily and quickly be used to move a medical wire by a predetermined movement amount, and can be used to efficiently perform various treatments inside a biological lumen.

According to an aspect of the disclosure, there is provided a medical device including a catheter including a first lumen into which a medical wire is insertable. The catheter possesses a distal end and a proximal end. The medical device further includes a tube-shaped body comprising a second lumen configured to communicate with the first lumen. The tube-shaped body possesses a distal end, and the distal end of the tube-shaped body is proximal of the proximal end of the catheter. The medical device also includes a delivery mechanism in the second lumen of the tube-shaped body, the delivery mechanism enabling a delivery operation to move the medical wire beyond the distal end of the catheter by a predetermined movement amount

According to another aspect of the disclosure, there is provided a medical device assembly including an elongated medical wire configured to penetrate a biological lumen and a medical device. The medical device includes a catheter including a first lumen, an elongated tube-shaped body proximal to the catheter and comprising a second lumen that communicates with the first lumen. The medical wire is insertable into the first lumen via the second lumen. The medical device also includes a delivery mechanism within the second lumen of the tube-shaped body configured to move the medical wire distally by a predetermined movement amount to penetrate the biological lumen. The medical wire is assembled to the medical device such that the medical wire is attachable to and detachable from the medical device. Another aspect of the disclosure here is a method that includes inserting a medical device assembly into a living body. The medical device assembly includes a lumen, a gripping member at least partially located in the lumen, and a penetrating wire within the lumen. The gripping member holds the penetrating wire in a fixed position so that the gripping member and the penetrating wire move together while the gripping member is gripping the penetrating wire. The method includes moving the medical device assembly to an occluded stenosed site in the living body, pushing the gripping member in a distal direction by a predetermined amount so that the penetrating wire moves distally by the predetermined amount to create a hole in the occluded stenosed site, and the gripping member automatically releasing the penetrating wire when the gripping member has moved the predetermined amount such that further distal movement of the gripping member does not move the penetrating wire.

According to the medical device configured as described above, the medical wire can be delivered to the distal side by the predetermined movement amount by operating the delivery mechanism accommodated in the second lumen of the tube-shaped body. In addition, the medical wire is moved by being provided with a mechanical operation force via the delivery mechanism of the medical device. Accordingly, the pressing force can be more satisfactorily transmitted to the distal side of the medical wire than when the medical wire is pressed or pulled by an operator's operation outside a living body. Therefore, various treatments using the medical wire can be efficiently performed in the biological lumen.

The medical device assembly configured as described above includes the medical wire and the medical device including the delivery mechanism which enables the medical wire to move to the distal side by the predetermined movement amount. The medical wire is attachable to and detachable from the medical device. The disclosed medical device assembly allows the medical wire to be properly replaced even while an operator deploys his or her operation technique. Therefore, various medical wires suitable for treatment conditions can be selectively used, improving usability.

### BRIEF DESCRIPTION OF THE DRAWINGS

FIG. 1 is an overall configuration diagram of a medical device assembly according to a first embodiment.

FIG. 2 is a sectional view on a side extending along a longitudinal direction of the medical device assembly according to the first embodiment.

FIGS. 3A to 3C are enlarged views of a proximal portion of the medical device assembly. FIGS. 3A to 3C illustrate an operation for delivering a penetrating wire to a distal side.

FIGS. 4A to 4D illustrate examples of the distal shape of the penetrating wire.

FIGS. 5A to 5C illustrate examples of a gripping member.

FIGS. 6A and 6B are enlarged sectional views showing the fixing operation of a fixing portion according to the first embodiment. FIG. 6A illustrates a state where the fixing portion is unfixed, and FIG. 6B illustrates the fixing portion in the fixed state.

FIGS. 7A to 7C are enlarged sectional views showing the delivery mechanism according to the first embodiment delivering the penetrating wire.

FIGS. 8A to 8C are enlarged sectional views showing a holding member holding the fixing portion in the unfixed state.

FIGS. 9A to 9C are enlarged sectional views showing a delivery return mechanism returning the delivered penetrating wire.

FIG. 10 is an overall configuration diagram of a medical device assembly according to a modification example of the first embodiment.

FIG. 11 is a sectional view on a side extending along the longitudinal direction of the medical device assembly according to the modification example of the first embodiment.

FIGS. 12A to 12C are views illustrating a delivery mechanism included in the medical device assembly according to the modification example of the first embodiment delivering a penetrating wire.

FIG. 13 is an overall configuration diagram of a medical device assembly according to a second embodiment.

FIG. 14 is a sectional view on a side extending along the longitudinal direction of a medical device assembly according to the second embodiment.

FIGS. 15A and 15B are enlarged sectional views illustrating the structure and operation of the fixing portion according to Modification Example 1.

FIGS. 16A and 16B are enlarged sectional views illustrating the structure and operation of the fixing portion according to Modification Example 2.

### DETAILED DESCRIPTION

Set forth below is a detailed description of embodiments of a medical device and a medical device assembly representing examples of the inventive medical device and medical device assembly disclosed here. The following description does not limit the technical scope or the definition of terms set forth in the appended claims. In addition, in some cases, dimensional ratios in the drawings are exaggerated for the convenience of explanation and thus may be different from actual ratios.

### First Embodiment

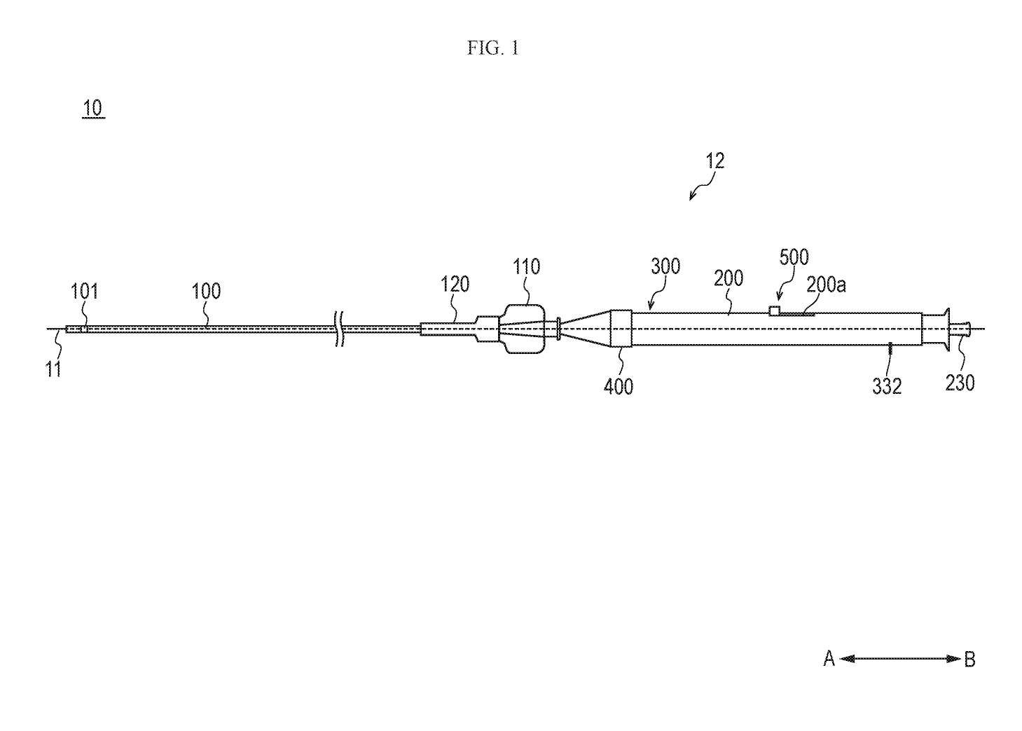

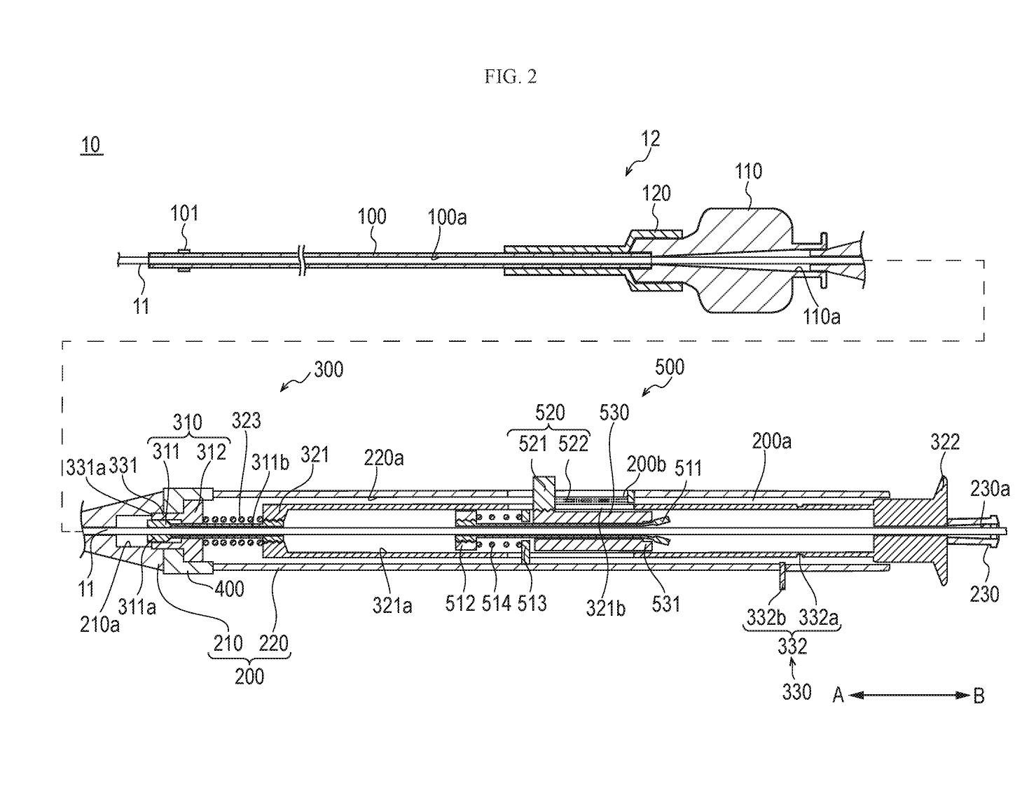

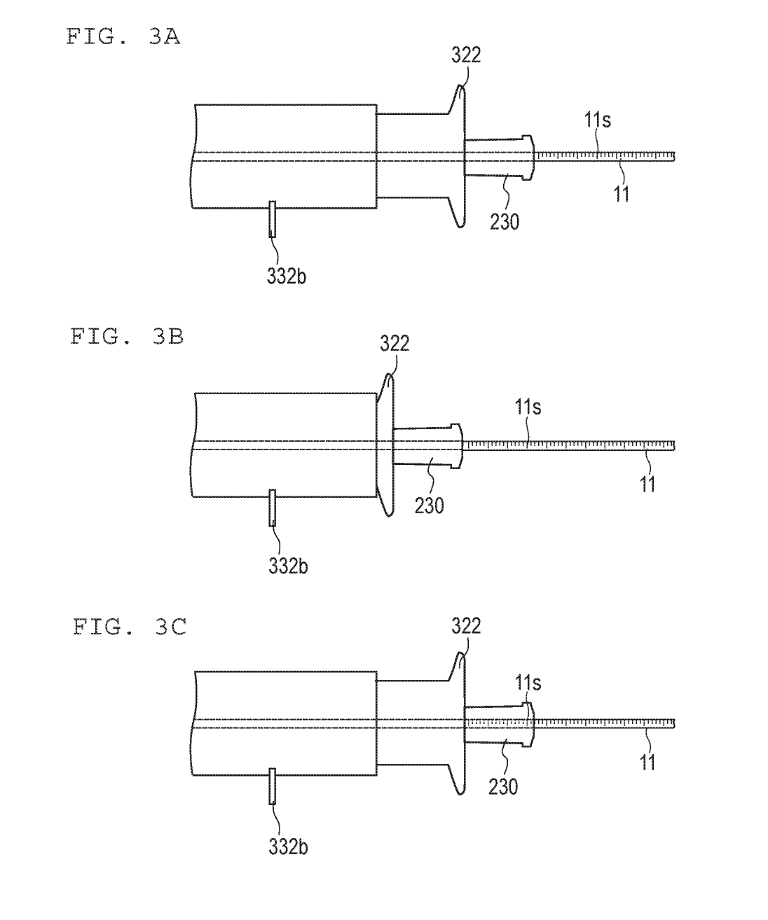

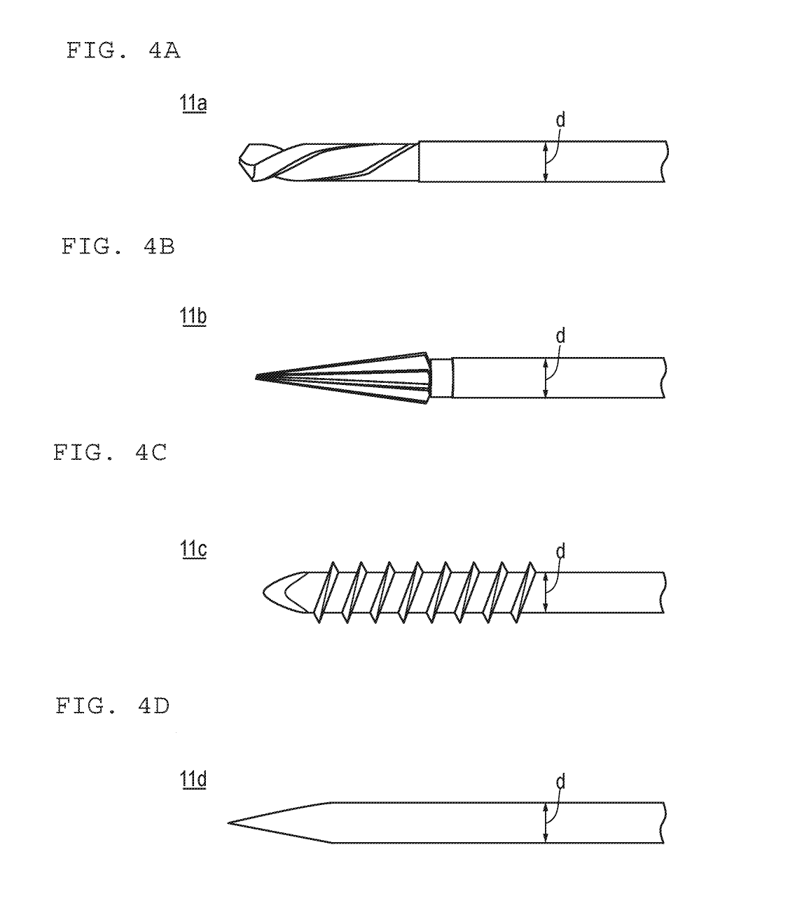

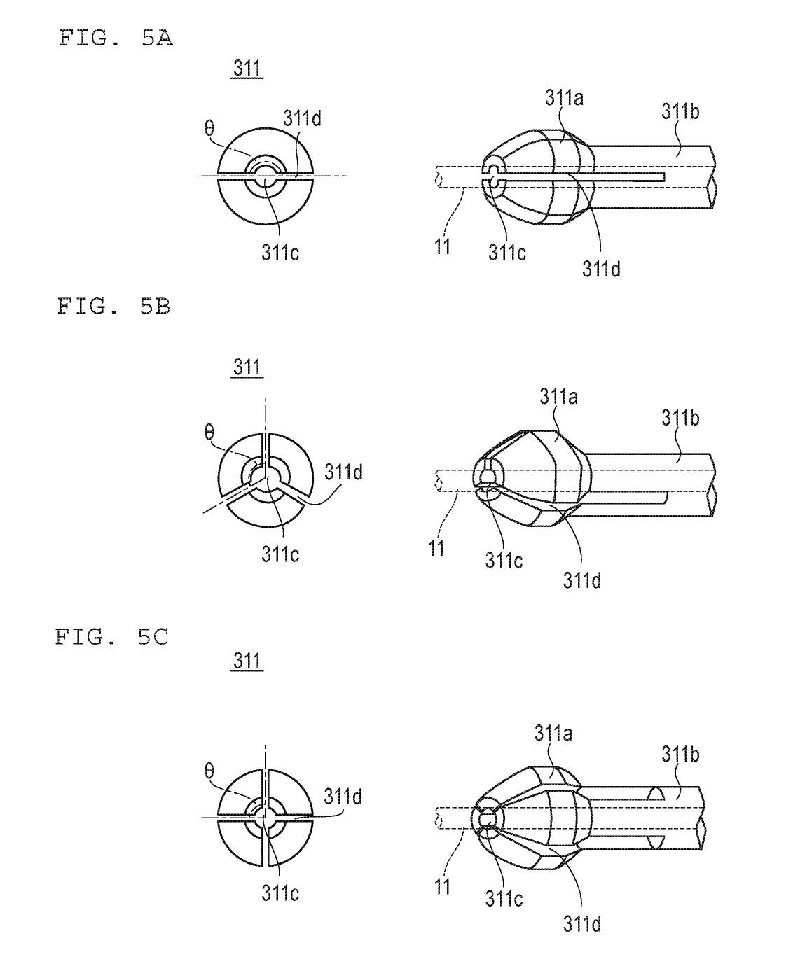

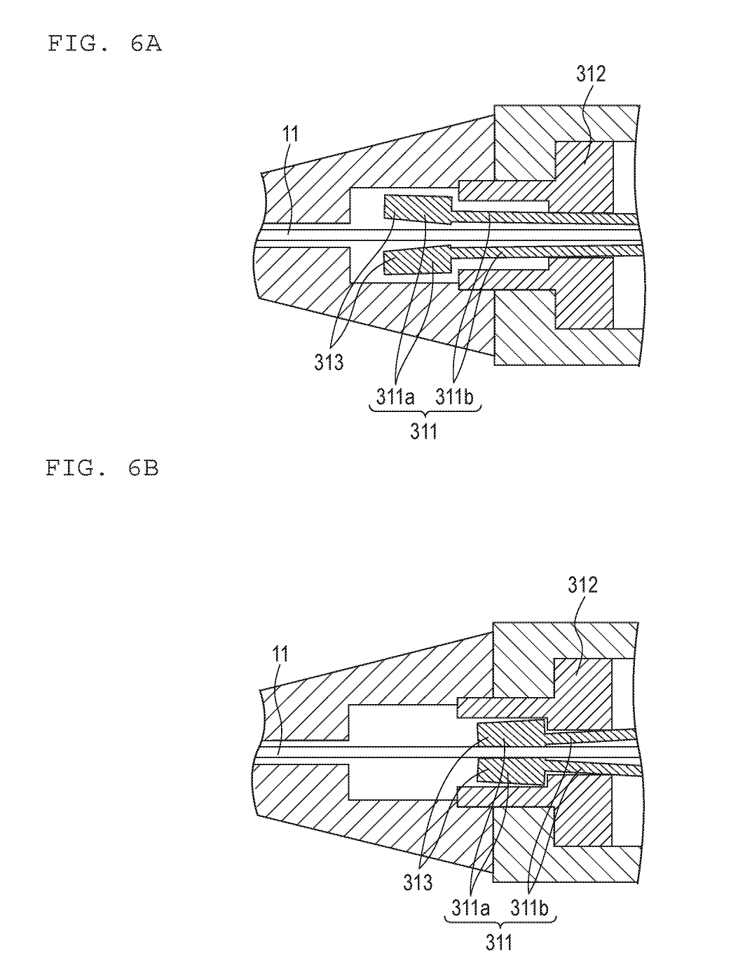

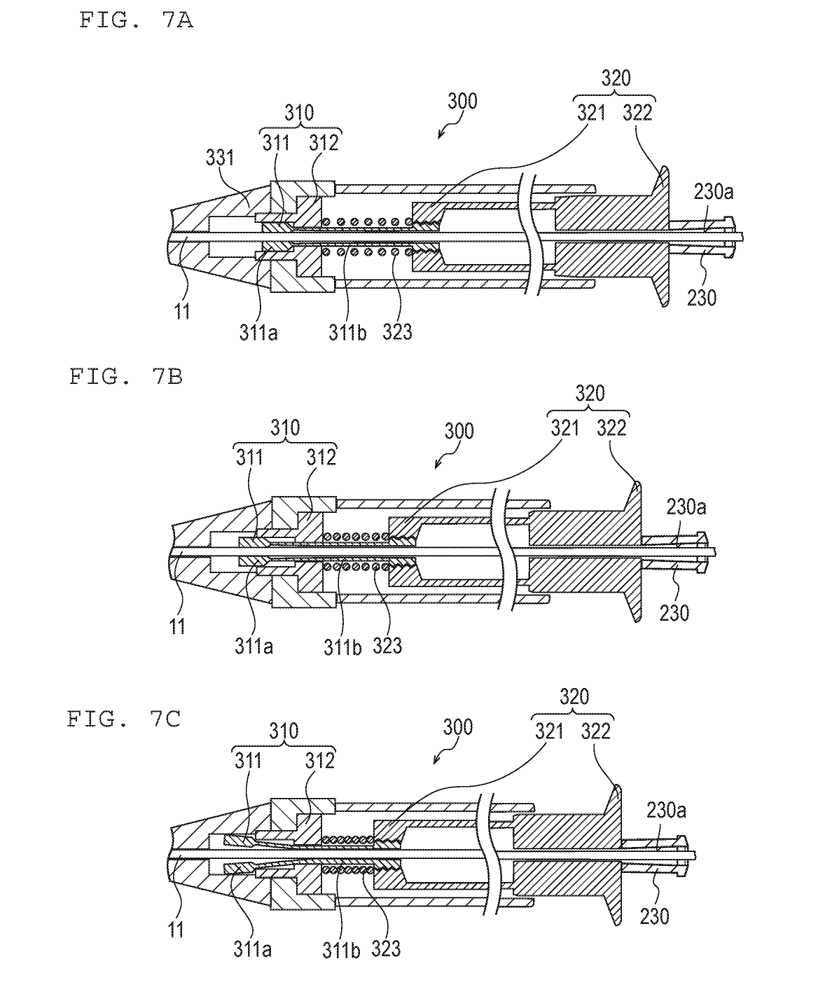

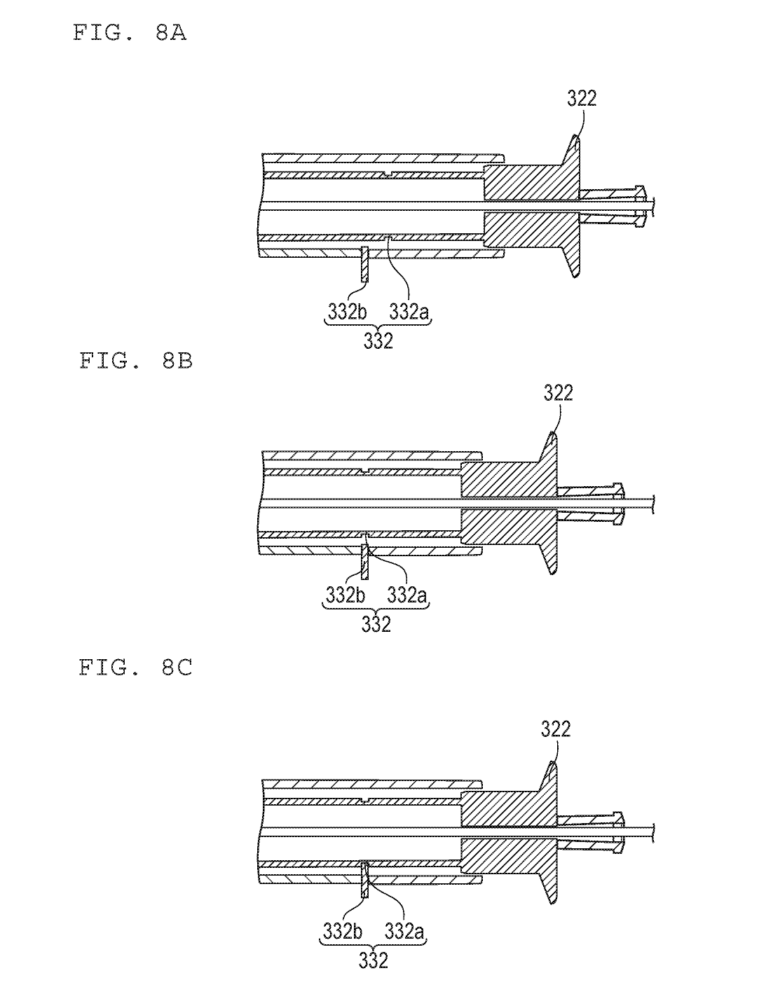

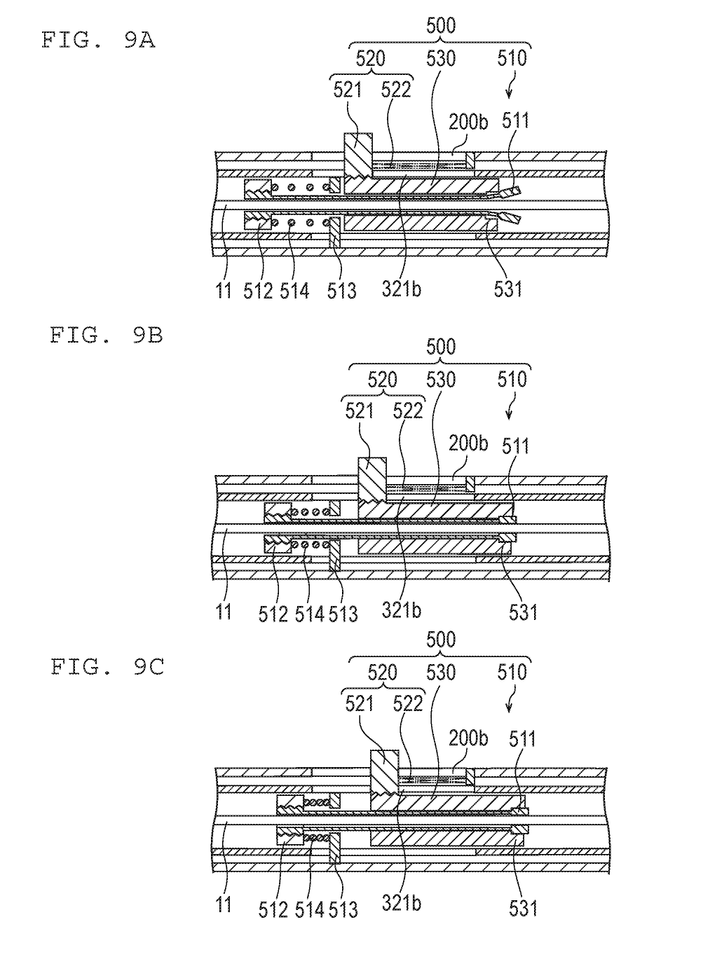

FIG. 1 is an overall configuration diagram of a medical device assembly 10 according to a first embodiment. FIG. 2 is a sectional view on a side extending along a longitudinal direction of the medical device assembly 10 according to the first embodiment. FIGS. 3A to 3C are enlarged views of a proximal portion of the medical device assembly 10, and illustrate an operation for delivering a penetrating wire 11 to a distal side. FIGS. 4A to 4D are views illustrating examples of the shape of the distal end of the penetrating wire 11 (corresponding to a medical wire 11). FIGS. 5A to 5C are views illustrating examples of a gripping member 311. FIGS. 6A and 6B are enlarged sectional views illustrating a fixing operation of a fixing portion 310 according to the first embodiment. FIGS. 7A to 7C are enlarged sectional views illustrating an operation in which a delivery mechanism 300 according to the first embodiment delivers the penetrating wire 11. FIGS. 8A to 8C are enlarged sectional views illustrating an operation in which a holding member 332 holds a state where the fixing portion 310 is unfixed. FIGS. 9A to 9C are enlarged sectional views illustrating an operation in which a delivery return mechanism 500 returns the delivered penetrating wire 11.

In the description below, a side inserted into a body lumen is referred to as the distal side or distal end (direction of an arrow A in the drawing), a side on which a tube-shaped body 200 is disposed, which serves as an operator's hand operation side, is referred to as the proximal side or proximal end (direction of an arrow B in the drawing), and a direction extending along an arrow A-B is referred to as the longitudinal direction (i.e., the axial direction).

As illustrated in FIG. 1, the medical device assembly 10 according to the first embodiment includes the elongated medical wire 11 to be inserted into a biological lumen. The medical wire 11 is assembled so as to be attachable to a medical device 12 and detachable from the medical device 12.

As shown in FIG. 2, the medical device 12 has a catheter 100 that has a lumen 100a into which the medical wire 11 is inserted (i.e., the medical wire 11 is insertable into and movable within the lumen 100a of the catheter 100). The medical device 12 also has a tube-shaped body 200 disposed on the proximal side of the catheter 100. The tube-shaped body 200 includes a lumen 200a which communicates with the lumen 100a. A delivery mechanism 300 is accommodated in the lumen 200a of the tube-shaped body 200 that enables a user to perform a delivery operation of moving the medical wire 11 towards the distal side by a predetermined movement amount (i.e., the medical wire 11 is moved distally by a specific distance). Furthermore, the medical device 12 includes a rotary operation unit 400 which rotates the medical wire 11, and a delivery return mechanism 500 which moves the medical wire 11 to the proximal side.

For example, the medical wire 11 may be a known guide wire used to guide a catheter device such as a balloon catheter and the like into a biological lumen, or a known penetrating wire used to penetrate a stenosed site formed in the biological lumen. In the present embodiment, the description will be in reference to using a penetrating wire as the medical wire 11.

The penetrating wire 11 is mainly used in order to penetrate the stenosed site (or an occluded site) in which a stenosis progresses to such an extent that the guide wire is less likely to be inserted (i.e., it is difficult to insert a non-penetrating wire into the stenosis site). The penetrating wire 11 penetrates the stenosed site to create a penetrating hole, thereby enabling the guide wire to be inserted into the stenosed site after the penetrating wire 11 is pulled back or retracted.

As illustrated in FIG. 3A, a proximal portion of the penetrating wire 11 has a scale 11s (marker) which indicates a movement amount of the penetrating wire 11 in the extending direction of the tube-shaped body 200.

The penetrating wire 11 has a distal shape (i.e., a shape at the distal end of the penetrating wire 11) to penetrate the stenosed site. The distal shape of the distal end of the penetrating wire 11 can be a drill shape illustrated in FIG. 4A, a reamer shape illustrated in FIG. 4B, a screw shape illustrated in FIG. 4C, a conical shape (tapered or pointed shape) illustrated in FIG. 4D, or the like.

When using a penetrating wire 11a having the drill shape illustrated in FIG. 4A, the penetrating wire 11a is rotated and moved forward to the distal side (i.e., moved distally). In this manner, penetrating work can be efficiently carried out to scrape or remove material from the stenosed site. When using a penetrating wire 11b having the reamer shape illustrated in FIG. 4B, it is possible to easily carry out work for opening a hole and broadening the hole in the stenosed site by using a blade portion disposed around a main body having a conical shape (i.e., the reamer shape is tapered or pointed). When using a penetrating wire 11c having the screw shape illustrated in FIG. 4C, the penetrating wire 11c is rotated and moved forward to the distal side (i.e., moved distally). In this manner, the penetrating wire 11c can more reliably penetrate the stenosed site along a helical thread of the penetrating wire 11c. When using a penetrating wire 11d having the conical shape (tapered shape) illustrated in FIG. 4D, the penetrating wire 11d can be easily inserted into the catheter 100 and can easily penetrate the stenosed site. Accordingly, it is possible to improve the pushing performance of the penetrating wire 11d into the biological lumen (i.e., it is easier to push the penetrating wire 11d in the distal direction).

The distal shape of the penetrating wire 11 is not limited to the shapes illustrated in FIGS. 4A to 4D, as long as the distal shape is provided with the ability to penetrate the stenosed site. For example, the respective penetrating wires 11a to 11d can be replaced and used when the same operation technique is used. Alternatively, various treatments can be progressively performed by replacing penetrating wires other than those which illustrated in FIGS. 4A to 4D or the guide wire.

A guide wire known in the medical field can be used as the guide wire that is inserted into the medical device 12. As an example, it is possible to use a guide wire possessing a substantially linear overall shape when a load is not applied on the guide wire, a guide wire possessing a shape with a curved distal side or distal end when a load is not applied on the guide wire, a guide wire which is coated with a polymer, or the like.

An outer diameter d of a linear portion (a portion on the proximal side further from the distal portion) of the respective penetrating wires 11a to 11d used for the medical device 12 according to the embodiment can be 0.3 mm to 1.0 mm, for example.

As illustrated in FIG. 2, the catheter 100 has the lumen 100a into which the penetrating wire 11 is inserted. The penetrating wire 11 is insertable over the entire length of the catheter 100 from the proximal end to the distal end. A hub 110 is connected to the proximal portion of the catheter 100. Any imaging marker 101 having X-ray contrast capability is at the distal portion of the catheter 100. A position of the distal portion of the catheter 100 can be clearly confirmed on an X-ray image by using the imaging marker 101. The imaging marker 101 can be configured to include a material provided with the X-ray contrast capability. For example, the imaging marker 101 preferably is a metal such as platinum, gold, silver, iridium, titanium, tungsten, or alloys of these metals.

A covering portion 120 covers a portion of the outer periphery of the hub 110. The covering portion 120 is connected to the proximal portion of the catheter 100. For example, the covering portion 120 can be a resin material such as a fluorine resin tube, rubber tube, and the like.

It is preferable to use a flexible material to form the catheter 100. For example, it is possible to use polyolefin such as polyethylene, polypropylene, and the like, polyester such as polyimide, polyethylene terephthalate, and the like, fluorine-based polymer such as ETFE and the like, resins such as PEEK, polyimide, and the like. In the above-described resins, a thermoplastic resin can be preferably used. In order to improve kink resistance or torque transmitting performance, the catheter 100 may employ a structure including a reinforcement body (not illustrated) obtained by braiding a metal wire into a net shape or a coil shape.

The hub 110 is connected to the proximal portion of the catheter 100 and the distal end of the tube-shaped body 200 in a liquid-tight manner. For example, the hub 110 material may be a synthetic resin such as polycarbonate, polyolefin, styrene resins, polyester, and the like, stainless steel, aluminum, aluminum alloys. For example, as the polyolefin, it is possible to use polyethylene, polypropylene, ethylene-propylene copolymers, and the like.

The tube-shaped body 200 has a distal member 210 at the distal portion of the tube-shaped body 200 and a main body portion 220 connected to the proximal end of the distal member 210. A side surface portion (i.e., the wall) of the tube-shaped body 200 has a slit 200b extending in the longitudinal direction, in order to form a movement path of an operation lever 521 of the delivery return mechanism 500 (described below).

The distal member 210 has an outer shape of a substantially truncated cone which tapers to the distal side (i.e., the distal member 210 is tapered from its proximal end to its distal end). The distal end of the distal member 210 is inserted into the proximal end of the hub 110 and is connected to the hub 110. The penetrating wire 11 is inserted into an insertion hole 210a extending to the distal end portion of the distal member 210.

The main body portion 220 has an elongated cylinder shape, and includes a lumen 220a into which the penetrating wire 11 is insertable. The shape of the main body portion 220 is not limited to a cylindrical shape and the main body portion 220 shape can be a triangular prism, a quadrangular prism, or the like.

The lumen 100a of the catheter 100 and the lumen 220a of the main body portion 220 communicate with each other via the insertion hole 210a of the distal member 210 and the lumen 110a inside the hub 110.

The proximal portion of the tube-shaped body 200 has a port 230 installed in order to supply various fluid such as a heparinized physiological salt solution, a physiological salt solution, and the like for flushing the inside of the medical device 12. A lumen 230a of the port 230 communicates with the lumen 200a of the tube-shaped body 200 and the lumen 100a of the catheter 100.

The tube-shaped body 200 material can have a relatively high rigidity and can be, for example, a resin or metal such as SUS and the like. The catheter 100, the hub 110, and the tube-shaped body 200 may be integrally configured by using a mechanical interlock structure in which respective members are interlocked with each other by means of fitting or the like, or in such a way that the respective members are fixed to each other by using an adhesive or the like.

As illustrated in FIGS. 2 and 7A to 7C, the delivery mechanism 300 includes the fixing portion 310 which fixes the penetrating wire 11 (i.e., holds the penetrating wire 11 in place), a moving unit 320 which moves the fixing portion 310 when the penetrating wire 11 is fixed to the distal side of the lumen 200a of the tube-shaped body 200 by a predetermined movement amount (i.e., the moving unit 320 moves the penetrating wire 11 distally by a predetermined amount), and an unfixing portion 330 which automatically unfixes the penetrating wire 11 fixed by the fixing portion 310 after the penetrating wire has moved the predetermined amount (i.e., the unfixing portion 330 allows the penetrating wire 11 to move proximally or distally relative to the catheter 100 after the penetrating wire has moved the predetermined amount).

The fixing portion 310 included in the delivery mechanism 300 has a gripping member 311 which grips the penetrating wire 11, and an engagement member 312 which is around the outer periphery of the proximal portion of the gripping member 311 when the fixing portion 310 is in the fixing position.

The moving unit 320 included in the delivery mechanism 300 has a pressing member 321. A user can press the pressing member 321 in the distal direction to press the gripping member 311 to move to the distal side of the lumen 200a, and a plunger portion 322 (i.e., a knock portion) in the proximal portion of the moving unit 320.

The unfixing portion 330 included in the delivery mechanism 300 includes a movement restriction member 331 (i.e., a movement restriction portion) disposed in the distal member 210 on the distal side of the engagement member 312, and a holding member 332 at the proximal portion of the pressing member 321.

The configuration and the operation of each unit of the delivery mechanism 300 is described below.

The gripping member 311 has a pinching piece 311a which pinches the penetrating wire 11, and a support portion 311b which is connected to the proximal end of the pinching piece 311a. The support portion 311b is fixed to the moving unit 320 and extends substantially parallel to the longitudinal direction of the tube-shaped body 200. The support portion 311b moves in the longitudinal direction along the extending direction of the tube-shaped body 200 (hereinafter, referred to as the “longitudinal direction”) in accordance with the movement of the moving unit 320 (i.e., distal or proximal movement of the moving unit 320).

Examples of the gripping member 311 will be described in reference to FIGS. 5A to 5C. In FIGS. 5A to 5C, the right view illustrates an overall perspective view of the gripping member 311, and the left view illustrates a front view when the gripping member 311 is viewed from the distal side.

As illustrated in FIG. 5A, the gripping member 311 according to the present embodiment includes an insertion hole 311c into which the penetrating wire 11 is insertable (i.e., the penetrating wire 11 is slidable through the insertion hole 311c). The distal portion of the gripping member 311 has two slits 311d which extend outward in a radial direction from the central axis of the gripping member 311 and which have a predetermined length from a distal surface of the gripping member 311. The respective slits 311d are formed so that a phase difference θ between the respective slits 311d is an angle of approximately 180 degrees (i.e., two distal slits 311d are spaced 180 degrees from one another).

The phase difference θ between the respective slits 311d is not limited to the angle of approximately 180 degrees. For example, the phase difference θ can be set to approximately 120 degrees as illustrated in FIG. 5B, or can be set to approximately 90 degrees as illustrated in FIG. 5C. An inner surface of the pinching piece 311a can be aligned with an outer shape of the penetrating wire 11 by decreasing the phase difference θ between the respective slits 311d (by increasing the number of slits 311d). Accordingly, the penetrating wire 11 can more reliably gripped by the gripping member 311, and a wider range of penetrating wire 11 shapes are available.

In addition, the slit 311d formed between the pinching pieces 311a included in the gripping member 311 also functions as a fluid circulating path. Therefore, a flushing solution such as the heparinized physiological salt solution, the physiological salt solution, and the like (which is fed from the port 230 via the lumen 200a of the tube-shaped body 200) can be circulated to the lumen 100a on the catheter 100 side through the slit 311d.

When the pinching piece 311a is opened, as illustrated in FIG. 6A, the gripping member 311 releases gripping (i.e., fixing) of the penetrating wire 11. On the other hand, as illustrated in FIG. 6B, when the pinching piece 311a is closed, the penetrating wire 11 is gripped by and fixed to the gripping member 311.

The pinching piece 311a has a tapered shape portion 313 (i.e., an adjusting portion 313) which adjusts a plane direction of a portion which is in contact with the penetrating wire 11 when the penetrating wire 11 is gripped, so as to be parallel to the extending direction of the penetrating wire 11 (i.e., the tapered shape portion 313 of the pinching piece 311a allows the contact portion of the pinching piece 311a that contacts/grips the penetrating wire 11 to remain parallel to the penetrating wire 11 throughout the gripping progression).

As illustrated in FIG. 6A, the tapered shape portion 313 tilts in the extending direction of the penetrating wire 11 when the penetrating wire 11 is not pinched, that is, in an open state. As illustrated in FIG. 6B, if the pinching piece 311a is pressed from the outside by the engagement member 312 and is brought into a closed state, the contact surface of the pinching piece 311a which comes into contact with the penetrating wire 11 is parallel to the extending direction of the penetrating wire 11. When the gripping member 311 pinches the penetrating wire 11, if the contact surface of the pinching piece 311a is disposed parallel to the extending direction of the penetrating wire 11, the area of the contact surface between the pinching piece 311a and the penetrating wire 11 can be increased (i.e., maximized). Therefore, it is possible to improve the gripping force applied by the gripping member 311 on the penetrating wire 11.

The material of the gripping member 311 is not particularly limited as long as the gripping member 311 can grip the penetrating wire 11. For example, it is possible to use a resin material, a metal material, or the like.

As illustrated in FIG. 2, the engagement member 312 has a ring shape and is disposed on the outer periphery of the gripping member 311 when the fixing portion 310 is in the fixing position (i.e., when the gripping member 311 grips the penetrating wire 11). The engagement member 312 engages with the gripping member 311, thereby inhibiting the gripping member 311 from spreading outward in the radial direction and maintaining a state where the gripping member 311 grips the penetrating wire 11. In a state where the gripping member 311 is closed, the engagement member 312 is fitted to the outer periphery of the gripping member 311, thereby inhibiting the pinching piece 311a from spreading outward in the radial direction (i.e., the pinching piece 311a will expand radially outward when not inhibited). In this manner, the gripping member 311 holds a state of gripping the penetrating wire 11.

The pressing member 321 included in the moving unit 320 is operable to press the gripping member 311 to move to the distal side of the lumen 200a. The pressing member 321 possesses an elongated cylindrical shape which extends from the distal end to the proximal end and is interlocked with the proximal portion of the gripping member 311. The outer diameter of the pressing member 321 is smaller than the inner diameter of the tube-shaped body 200, and is configured so that the pressing member 321 is movable relative to the lumen 200a of the tube-shaped body 200 in the longitudinal direction.

The plunger portion 322 included in the moving unit 320 is integrally disposed in the proximal portion of the pressing member 321. The plunger portion 322 extends in the longitudinal direction and protrudes from the proximal end of the tube-shaped body 200 (i.e., the plunger portion 322 extends proximally relative to the proximal end of the tube-shaped body 200). The plunger portion 322 is configured to be pressed and operated from the outside by a user. The plunger portion 322 possesses a cross-sectional shape that has a larger outer diameter than the outer diameter of the tube-shaped body 200 in order to facilitate the user's pressing. The pressing member 321 is moved to the distal side by pressing the plunger portion 322 (i.e., the pressing member 321 moves distally when pressed by a user).

A first elastic member 323 such as a coil spring and the like is disposed between the pressing member 321 and the engagement member 312. The first elastic member 323 biases the pressing member 321 to the proximal side. In this manner, a configuration is adopted in which the pressing member 321 returns to its initial position if the pressing force is released after the pressing member 321 is moved to the distal side.

The distance that the penetrating wire (or the guide wire) moves to the distal side is not particularly limited. However, for example, the penetrating wire is set to have a moving distance (i.e., a predetermined movement amount) of 0.3mm to 3.0 mm by pressing the plunger portion 322.

The first elastic member 323 material is not particularly limited as long as the material has elasticity to bias the gripping member 311 in the proximal direction. For example, it is possible to use metal such as stainless steel, aluminum, copper, iron, nickel titanium, and the like, or resins. In addition, the shape of the first elastic member 323 is not limited to a coil spring shape and may be an elastic porous member such as a leaf spring and a sponge.

In the movement restriction member 331, the insertion hole 210a of the distal member 210 has a decreased diameter portion 331a whose diameter decreases inward in the radial direction. The inner diameter of the decreased diameter portion 331a is smaller than of the inner diameter of the engagement member 312, and the decreased diameter portion 331a abuts the engagement member 312. If it is attempted to move the engagement member 312 to the distal side of the decreased diameter portion 331a, the engagement member 312 abuts against the decreased diameter portion 331a and movement to the distal side is restricted (i.e., the engagement member 312 cannot be moved distally beyond the decreased diameter portion 331a). The movement restriction member 331 thus restricts the distal movement of the engagement member 312 when the gripping member 311 moves distally more than a predetermined amount, thereby causing the gripping member 311 and the engagement member 312 to disengage from each other.

The holding member 332 includes a locking groove 332a on an outer surface of the proximal portion of the pressing member 321 and a locking member 332b at a side surface portion of the tube-shaped body 200. The locking member 332b is configured to move radially inward to be locked in the locking groove 332a when the locking member 332b and the locking groove 332a are aligned in the longitudinal direction (i.e., axially aligned).

As illustrated in FIG. 2, when the gripping member 311 grips the penetrating wire 11, the locking groove 332a is disposed on the proximal side of the locking member 332b (i.e., the locking groove 332a is proximal to the locking member 332b). The locking groove 332a and the locking member 332b are disposed so that the locking groove 332a and the locking member 332b are located at the same position in the longitudinal direction when the gripping member 311 is pressed to the distal side by the pressing member 321 and the gripping member 311 releases the penetrating wire 11.

The locking member 332b has a rod shape extending in the radial direction of the tube-shaped body 200 and protruding from the outer surface of the tube-shaped body 200. The locking member 332b is thus configured to be movable inward in the radial direction by a pressing operation from the outside. When the gripping member 311 releases the penetrating wire 11, and the locking groove 332a and the locking member 332b are disposed at the same position (i.e., are axially aligned), the locking member 332b is configured to be pressed inward in the radial direction by a user, thereby enabling the locking groove 332a and the locking member 332b to be fitted to each other. In the illustrated embodiment, the gripping member 311 releases the penetrating wire 11 when the pressing member 321 is completely pressed (i.e., is moved distally until the pressing member 321 cannot move further in the distal direction). That is, the holding member 332 allows the locking groove 332a and the locking member 332b to be fitted to each other (i.e., fixed) to hold the pressing member 321 at the completely pressed position.

Next, the rotary operation unit 400 will be described.

The rotary operation unit 400 is a rotation force providing unit to rotate the penetrating wire 11 when the rotary operation unit 400 is rotated by a user's fingers gripping the rotatory operation unit 400 from the outside of the medical device 12. The rotary operation unit 400 is in contact with the outer surface of the engagement member 312, and is rotatable around the longitudinal axis (i.e., serving as the rotation axis). The rotary operation unit 400 uses a friction force generated with the engagement member 312 to transmit the rotation force to the engagement member 312. A mechanism for transmitting the rotation force is not limited to this friction force configuration. For example, the rotation force may be transmitted by disposing engagement teeth between the rotary operation unit 400 and the engagement member 312.

As illustrated in FIG. 2, if the user rotates the rotary operation unit 400 while the gripping member 311 grips the penetrating wire 11, the rotation force is transmitted to the engagement member 312. If the engagement member 312 is rotated, the rotation force is transmitted to the pinching piece 311a fitted to the engagement member 312, thereby pivoting (i.e., rotating) the penetrating wire 11 gripped by the gripping member 311. In this manner, the rotary operation unit 400 can rotate the penetrating wire 11 around the longitudinal axis serving as the rotation axis. While the penetrating wire 11 is moved forward, the penetrating wire 11 can be rotated by the rotary operation unit 400. Therefore, the penetrating wire 11 can more efficiently penetrate the stenosed site.

Next, the delivery return mechanism 500 will be described.

The delivery return mechanism 500 has a delivery return fixing portion 510 to fix the penetrating wire 11, a delivery return moving unit 520 to move the delivery return fixing portion 510 to the proximal side of the lumen 200a by a predetermined movement amount when the penetrating wire 11 is fixed, and a delivery return unfixing portion 530 to unfix the penetrating wire 11 fixed by the delivery return fixing portion 510.

The delivery return fixing portion 510 has a delivery return gripping member 511 (which includes the same configuration as the gripping member 311 of the delivery mechanism 300), a sliding member 512 configured to move in the longitudinal direction relative to the lumen 321a of the pressing member 321, a ring-shaped member 513 fixed to the tube-shaped body 200, and a second elastic member 514 between the sliding member 512 and the ring-shaped member 513. The sliding member is on the distal side of the delivery return gripping member 511.

The delivery return gripping member 511 grips and fixes the penetrating wire 11, and unfixes the penetrating wire 11 by releasing the grip. In the unfixed state, the delivery return gripping member 511 has a tapered shape in which the inner diameter and the outer diameter of the delivery return gripping member 511 increase from the distal end to the proximal end. In this manner, when the penetrating wire 11 is inserted from the port 230, the distal portion of the penetrating wire 11 can be smoothly guided into the central axis (delivery position) of the insertion hole 210a of the distal member 210 of the tube-shaped body 200. The pressing member 321 of the delivery mechanism 300 may also be provided with a function to guide the distal portion of the penetrating wire 11 to the delivery position by forming the pressing member 321 into the tapered shape in which the diameter of the proximal portion increases from the distal end to the proximal end.

The delivery return moving unit 520 includes an operation lever 521 which is interlocked with the delivery return gripping member 511. The operation lever 521 extends in the radial direction by protruding from the side surface portion of the tube-shaped body 200 (i.e., the operation lever 521 extends radially outwardly and protrudes beyond the outer diameter of the tube-shaped body 200). The slit 200b and the slit 321b are respectively disposed in the side surface portion of the tube-shaped body 200 and the side surface portion of the pressing member 321, thereby creating a movement path in which the operation lever 521 can move (i.e., be operated) in the longitudinal direction.

The delivery return unfixing portion 530 has a delivery return engagement member 531 which includes the same configuration as the engagement member 312 of the delivery mechanism 300. The delivery return engagement member 531 engages with the delivery return gripping member 511 to cause the gripping member 511 to grip or fix the penetrating wire 11. On the other hand, the delivery return engagement member 531 disengages from the delivery return gripping member 511 to release the penetrating wire 11 from the gripped state.

A third elastic member 522 is disposed between the proximal side of the operation lever 521 and the end portion on the proximal side of the slit 200b of the tube-shaped body 200. The third elastic member 522 is configured in this manner to return the operation lever 521 and the delivery return unfixing portion 530 to their initial positions when the pressing force is released after the operation lever 521 is moved to the distal side (i.e., the third elastic member 522 biases the operation lever 521 in the distal direction).

An operation is described below in reference to FIGS. 7A to 7C when the delivery mechanism 300 delivers the penetrating wire 11.

In an initial state before treatment using the penetrating wire 11 is performed, the outer periphery of the gripping member 311 included in the delivery mechanism 300 is fitted to the engagement member 312 as illustrated in FIG. 7A (i.e., the outer surface of the gripping member 311 contacts and is held within the inner diameter surface of the engagement member 312). The pinching piece 311a is thus restrained and is held in a closed state (gripped state). In this closed state, the penetrating wire 11 is fixed to the gripping member 311.

Subsequently, if a user (operator or the like) presses the plunger portion 322 distally to move the pressing member 321 to the distal side of the tube-shaped body 200, the first elastic member 323 compresses and the pressing member 321 moves to the distal side as illustrated in FIG. 7B. In this manner, the gripping member 311 interlocked with the pressing member 321 moves to the distal side. At this time, the outer periphery of the gripping member 311 is fitted to (i.e., in contact with) the engagement member 312. Accordingly, the pinching piece 311a of the gripping member 311 is restrained and is held in the closed state (gripped state). The penetrating wire 11 is fixed to the gripping member 311. Accordingly, the penetrating wire 11 moves to the distal side (i.e., moves distally) corresponding to the movement amount by which the gripping member 311 moves.

Thereafter, if the pressing member 321 is further moved to the distal side, the engagement member 312 abuts against the movement restriction member 331, and the movement of the engagement member 321 to the distal side is restricted. Accordingly, the gripping member 311 moves to the distal side further from the engagement member 312 (i.e., the gripping member 311 moves distally beyond the distal end of the engagement member 312). Therefore, as illustrated in FIG. 7C, the gripping member 311 is released from the restraint of the engagement member 312 and the gripping member 311 no longer applies the gripping force to grip the penetrating wire 11. In this manner, the penetrating wire 11 is no longer moved by the gripping member 311. Therefore, the fixing portion 310 is brought into a state where the gripping member 311 releases the grip of the penetrating wire 11. At this time, the fixing portion 310 is in a state where the penetrating wire 11 is unfixed. Accordingly, an operation for replacing the penetrating wire 11 can be performed. Therefore, for example, it is possible to easily individually replace the penetrating wire 11 with a guide wire after the penetrating wire 11 has completely penetrated the stenosed site.

If a user releases the plunger portion 322, the pressing member 321 is pressed to the proximal side by the first elastic member 323 (i.e., the first elastic member 323 moves the pressing member 321 proximally). In this manner, the pressing member 321 moves to the proximal side. Accordingly, the gripping member 311 interlocked with the pressing member 321 moves proximally and returns to its initial state illustrated in FIG. 7A.

According to the above-described operations, the delivery mechanism 300 can perform the delivery operation for moving the penetrating wire 11 to the distal side by a predetermined movement amount. If this operation is repeatedly performed, the penetrating wire 11 is enabled to be delivered stepwise by a constant amount (i.e., the penetrating wire 11 can be incrementally moved distally by repeating the operation). Accordingly, the movement amount of the penetrating wire 11 can be suitably adjusted. In addition, the penetrating wire 11 is moved by applying a mechanically actuating force via the delivery mechanism 300 on the distal side further from the operator's hand operation side (i.e., the force applied to move the penetrating wire 11 is at the gripping member 311, which is distal of where the operator's hand operation occurs). Therefore, compared to when the penetrating wire 11 is pressed and pulled by the operator's hand operation outside the living body, the pressing force can be satisfactorily transmitted to the distal portion of the penetrating wire 11. Furthermore, the penetrating wire 11 is delivered to the distal side when the penetrating wire 11 is supported by a tube wall of the insertion hole 210a included in the tube-shaped body 200. Accordingly, the pressing force can be more easily transmitted to the distal portion of the penetrating wire 11. Therefore, it is possible to efficiently perform treatment for penetrating the stenosed site by using the penetrating wire 11.

In the operation for delivering the above-described penetrating wire 11, the scale 11s is on the proximal portion (on the operator's hand operation side) of the penetrating wire 11. The operator can check the scale 11s while an operator deploys his or her operation technique. In this manner, it is possible to more accurately adjust the movement amount of the penetrating wire 11 in the extending direction of the tube-shaped body 200. Specifically, as illustrated in FIG. 3A, the penetrating wire 11 is first brought into its initial state before the treatment using the penetrating wire 11 is performed (i.e., before the penetrating wire is deployed from the medical device 12). Subsequently, if the plunger portion 322 is pressed as illustrated in FIG. 3B, the penetrating wire 11 moves to the distal side (i.e., towards the distal end of the medical device 12) together with the plunger portion 322. If the force to press the plunger portion 322 is later released, the plunger portion 322 returns to its initial state, and the port 230 moves to the proximal side in a state where the penetrating wire 11 stops as illustrated in FIG. 3C. At this time, a position of the proximal end of the port 230 is confirmed by using the scale 11s of the penetrating wire 11. In this manner, it is possible to confirm the movement amount of the penetrating wire 11 in the extending direction of the tube-shaped body 200.

Next, referring to FIG. 8, an operation is described below in which the holding member 332 holds a state where the fixing portion 310 is unfixed.

When the delivery mechanism 300 does not perform the delivery operation of the penetrating wire 11, for example, when the plunger portion 322 is not pressed, the locking groove 332a of the holding member 332 is located on the proximal side of the locking member 332b (i.e., the locking groove 332a is proximal of the locking member 332b in the longitudinal direction).

If a user completely presses the plunger portion 322 to move the pressing member 321 in the distal direction, as illustrated in FIG. 7C and described above, and the pressing member 321 is moved to the distal side until the fixing portion 310 releases the penetrating wire 11 gripped by the gripping member 311, the locking groove 332a and the locking member 332b are disposed at the same position in the axial direction as illustrated in FIG. 8B.

While a state of pressing the plunger portion 322 is maintained (i.e., the user continues to apply a force on the plunger portion in the distal direction), as illustrated in FIG. 8C, the locking member 332b is pressed inward in the radial direction to be locked in the locking groove 332a. Through this operation, the holding member 332 can maintain the gripping member 311 in an unfixed state (i.e., the holding member 332 keeps the gripping member 311 from gripping the penetrating wire 11). Since the unfixed state is held, the penetrating wire 11 can be replaced without the user having to press/hold the plunger portion 322. Therefore, it is possible to more easily carry out work for replacing the penetrating wire 11 with the guide wire.

Referring to FIG. 9, an operation is described below in which the delivery return mechanism 500 returns (i.e., retracts) the delivered penetrating wire 11.

At first, as illustrated in FIG. 7C, the fixing portion 310 included in the delivery mechanism 300 is unfixed from the penetrating wire 11 (i.e., the fixing portion 310 does not grip the penetrating wire 11). As illustrated in FIG. 9A, the delivery return fixing portion 510 included in the delivery return mechanism 500 is also unfixed from the penetrating wire 11 (i.e., the delivery return fixing portion 510 does not grip the penetrating wire 11).

Next, if the user moves the operation lever 521 to the proximal side, as illustrated in FIG. 9B, the operation lever 521 moves proximally and compresses the third elastic member 522. In this manner, the delivery return engagement member 531 interlocked with the operation lever 521 moves to the proximal side of the tube-shaped body 200. The delivery return engagement member 531 becomes fitted to the outer periphery of the delivery return gripping member 511, thereby causing the delivery return gripping member 511 to grip the penetrating wire 11.

Thereafter, if the operation lever 521 is further moved to the proximal side, the penetrating wire 11 moves proximally by a movement amount of the delivery return gripping member 511 as illustrated in FIG. 9C. The penetrating wire 11 moves proximally when the operation lever 521 moves in this manner because the penetrating wire 11 is gripped by and fixed to the delivery return gripping member 511.

If the user releases the force to press the operation lever 521, the operation lever 521 is pressed to the distal side by the third elastic member 522. When the operation lever 521 returns to its distal-most position in this manner, the delivery return gripping member 511 is pressed to the distal side by the second elastic member 514 disposed on the proximal side of the sliding member 512. In this manner, the penetrating wire 11 returns to its initial state illustrated in FIG. 9A.

According to the above-described operations, the delivery return mechanism 500 can perform the delivery return operation for moving the penetrating wire 11 to the proximal side by a predetermined movement amount. This operation is repeatedly performed, thereby enabling the delivered penetrating wire 11 to return stepwise by a constant amount (i.e., repeated operations of moving the penetrating wire 11 by a predetermined movement amount in the proximal direction allow the penetrating wire to be retracted).

As described above, the medical device 12 according to the present embodiment has the catheter 100 with the lumen 100a into which the penetrating wire 11 is inserted and the tube-shaped body 200 disposed on the proximal side further from the catheter 100 (i.e., proximal to the proximal end of the catheter 100). The tube-shaped body 200 includes the lumen 200a which communicates with the lumen 100a. The medical device 12 further includes the delivery mechanism 300 that is accommodated in the lumen 200a of the tube-shaped body 200 and that enables the delivery operation for moving the penetrating wire 11 to the distal side (i.e., distally beyond the distal end of the catheter) by the predetermined movement amount.

According to the medical device 12 configured in the described way, the penetrating wire 11 can be delivered to the distal side by the predetermined movement amount by operating the delivery mechanism 300 accommodated in the lumen 200a of the tube-shaped body 200. In addition, the penetrating wire 11 is moved by applying a mechanically actuating force via the delivery mechanism 300 of the medical device 12. Therefore, compared to a case where the penetrating wire 11 is pressed and pulled by the operator's hand operation outside the living body, the pressing force can be satisfactorily transmitted to the distal portion of the penetrating wire 11. Therefore, it is possible to efficiently penetrate the stenosed site inside the biological lumen by using the penetrating wire 11 (e.g., to form a penetrating hole in the occluded stenosed site).

In addition, the delivery mechanism 300 included in the medical device 12 according to the present embodiment has the fixing portion 310 which fixes the penetrating wire 11, the moving unit 320 which moves the fixing portion 310 in a state of fixing the penetrating wire 11 to the distal side of the lumen 200a with the predetermined movement amount, and the unfixing portion 330 which unfixes the penetrating wire 11 fixed by the fixing portion 310.

According to the medical device 12 configured in this way, the actuating force to move the penetrating wire 11 forward (i.e., distally) is generated at a position close to the distal portion of the penetrating wire 11 (i.e., the actuating force is applied nearer to the distal portion of the penetrating wire than where a force is applied by an operator). Accordingly, the pressing force can be effectively applied to the stenosed site from the distal portion of the penetrating wire 11, and the penetrating wire 11 can efficiently penetrate the stenosed site.

In addition, the fixing portion 310 included in the medical device 12 according to the present embodiment has the gripping member 311 which grips the penetrating wire 11, and the engagement member 312 which engages with the gripping member 311 to inhibit the gripping member 311 from spreading outward in the radial direction and to maintain a state where the gripping member 311 grips the penetrating wire 11. The moving unit 320 has the pressing member 321 which presses the gripping member 311 to move to the distal side of the lumen 200a (i.e., the gripping member 311 moves distally). The unfixing portion 330 has the movement restriction member 331 which causes the gripping member 311 and the engagement member 312 to disengage from each other by restricting the distal movement of the engagement member 312 when the gripping member 311 moves to the distal side more than the predetermined movement amount.

According to the medical device 12 configured in this way, the penetrating wire 11 can be more reliably gripped by the gripping member 311 and the engagement member 312 which are included in the fixing portion 310. In addition, in a state where the penetrating wire 11 is gripped, the penetrating wire 11 can be moved when the pressing member 321 is pressed to move distally. Therefore, the delivery operation is facilitated.

In addition, the fixing portion 310 included in the medical device 12 according to the present embodiment has the tapered shape portion 313 which adjusts a plane direction of the contact portion (i.e., the portion that contacts/grips the penetrating wire) when the penetrating wire 11 is fixed, so as to be parallel to the ,extending direction of the penetrating wire 11.

According to the medical device 12 configured in this way, the contact surface area between the fixing portion 310 and the penetrating wire 11 can be increased. Therefore, it is possible to improve the gripping force applied by the gripping member 311.

In addition, the medical device 12 according to the present embodiment further includes the rotary operation unit 400 which rotates the penetrating wire 11 around the rotation axis of the longitudinal axis extending along the extending direction of the tube-shaped body 200.

The rotary operation unit 400 allows the penetrating wire 11 to be rotated while the penetrating wire 11 is moved forward. Therefore, the penetrating wire 11 can more efficiently penetrate the stenosed site.

In addition, the medical device 12 according to the present embodiment further has the delivery return mechanism 500 which moves the penetrating wire 11 to the proximal side (i.e., moves the penetrating wire 11 proximally to retract the penetrating wire 11). The delivery return mechanism 500 has the delivery return fixing portion 510 which fixes the penetrating wire 11, the delivery return moving unit 520 which moves the delivery return fixing portion 510 in a state of fixing the penetrating wire 11 to the proximal side of the lumen 200a by a predetermined movement amount, and the delivery return unfixing portion 530 which unfixes the penetrating wire 11 after the penetrating wire 11 is fixed by the delivery return fixing portion 510.

According to the medical device 12 configured in this way, the delivered penetrating wire 11 can return to the proximal side with the predetermined movement amount by operating the delivery return mechanism 500 (i.e., the delivery return mechanism 500 can retract the penetrating wire 11 in the proximal direction by a predetermined movement amount). Therefore, the movement amount of the penetrating wire 11 can be finely adjusted, thereby improving operability.

In addition, the medical device assembly 10 according to the present embodiment includes the elongated penetrating wire 11 that is insertable into the biological lumen and the medical device 12. The penetrating wire 11 is assembled to be attachable to and detachable from the medical device 12.

According to the medical device assembly 10 configured in this way, the medical wire 11 can be properly replaced even while an operator deploys his or her operation technique. Therefore, various penetrating wires 11 suitable for treatment conditions can be selectively used, improving usability.

In addition, the penetrating wire 11 of the medical device assembly 10 according to the present embodiment has a distal shape (i.e., the penetrating wire is shaped at its distal end) with an ability to penetrate the stenosed site inside the biological lumen.

According to the medical device assembly 10 configured in this way, it becomes easier to penetrate the stenosed site. Therefore, the operation time can be shortened.

In addition, the penetrating wire 11 has the scale 11s which indicates the movement amount in the extending direction of the tube-shaped body 200.

According to the medical device assembly 10 configured in this way, it is possible to more accurately adjust the movement amount of the penetrating wire 11 in the extending direction of the tube-shaped body 200 by checking the scale 11s disposed in the penetrating wire 11 while an operator deploys his or her operation technique.

### MODIFICATION EXAMPLE OF FIRST EMBODIMENT

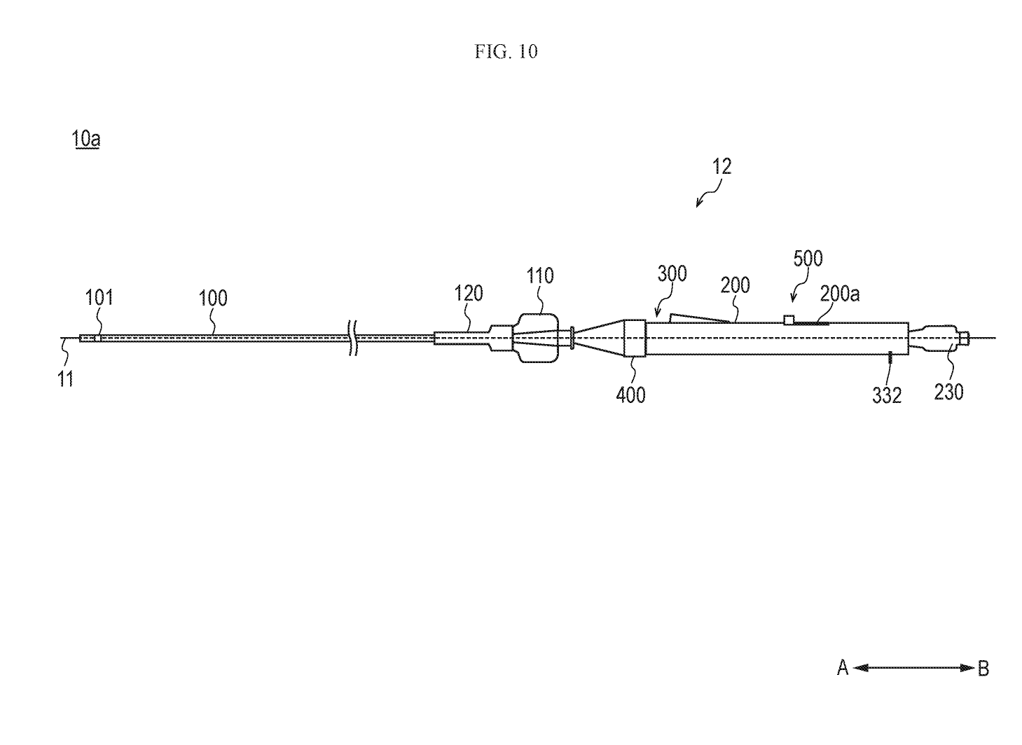

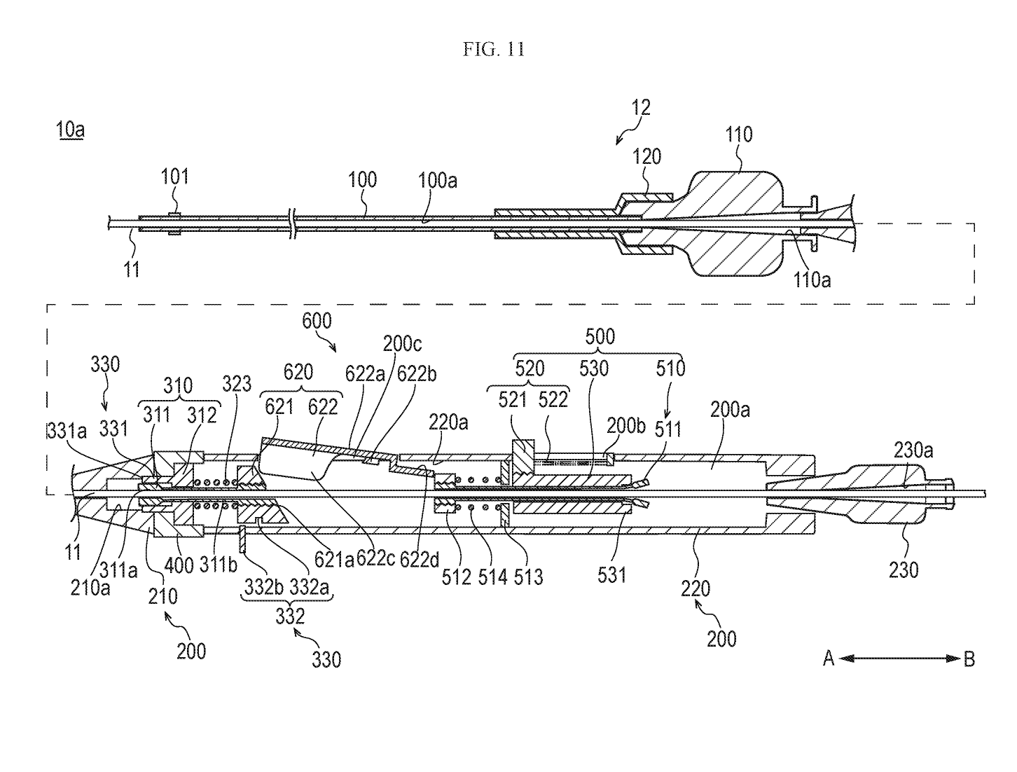

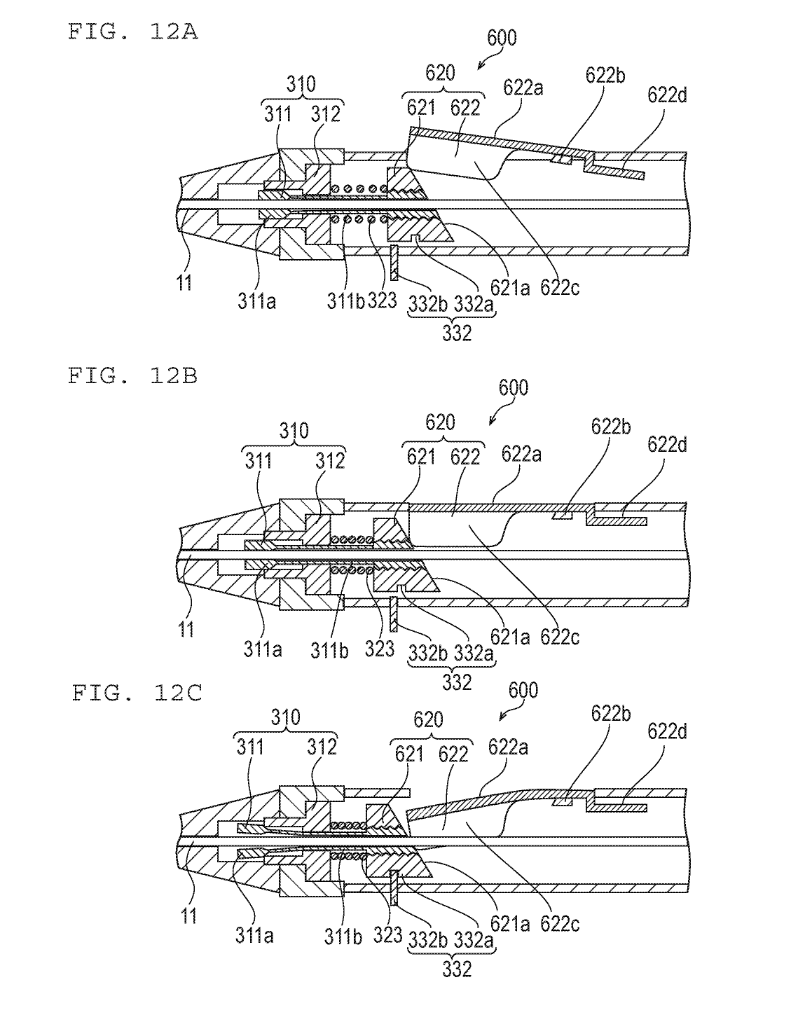

FIG. 10 is an overall configuration diagram of a medical device assembly 10a according to a modification example of the first embodiment. FIG. 11 is a sectional view on a side extending along the longitudinal direction of the medical device assembly 10a according to the modification example of the first embodiment. FIGS. 12A to 12C are views illustrating an operation using a delivery mechanism 600 included in the medical device assembly 10a according to the modification example of the first embodiment delivers the penetrating wire 11. The same reference numerals are used for configurations which are the same as those according to the first embodiment, and thus, description of the configurations which are the same will be omitted.

Unlike the first embodiment, the medical device assembly 10a according to the modification example of the first embodiment includes a moving unit 620 included in the delivery mechanism 600. Other configurations are substantially the same as those according to the first embodiment.

The delivery mechanism 300 according to the above-described first embodiment adopts a configuration in which the plunger portion 322 is pressed from the proximal end to the distal end (i.e., the plunger portion 322 is pressed towards the distal end) to move the penetrating wire 11 fixed by the fixing portion 310 to the distal side (as illustrated in FIGS. 7A to 7C). On the other hand, the delivery mechanism 600 according to the present modification embodiment adopts a configuration in which a plunger portion 622 is pressed inward from the outside in the radial direction of the tube-shaped body 200 to move the penetrating wire 11 fixed by the fixing portion 310 to the distal side (i.e., a user presses the plunger portion 622 radially inward to move the penetrating wire 11 distally).

In brief, referring to FIGS. 10 and 11, the moving unit 620 included in the delivery mechanism 600 has a pressing member 621 which is movable to press the gripping member 311 to move the gripping member 311 to the distal side of the lumen 200a. The plunger portion 622 is disposed in the side surface portion (i.e., the wall) of the tube-shaped body 200. The plunger portion 622 is easily operated by the user's finger. Therefore, it is possible to further improve operability.

An opening portion 200c is disposed in the side surface portion of the tube-shaped body 200, and the plunger portion 622 can protrude from the side surface portion of the tube-shaped body 200. In other words, the wall of the tube-shaped body includes an opening 200c, and the plunger portion 622 protrudes radially outward from the opening 200c.

The configuration and operation of each portion in the moving unit 620 included in the delivery mechanism 600 is described below.

As illustrated in FIG. 11, the pressing member 621 has a proximal surface 621a which tilts in the radial direction of the tube-shaped body 200.

The plunger portion 622 includes an operation unit 622a which is pressed and operated by a user from the outside of the tube-shaped body 200, a fulcrum portion 622b which supports an inner surface when being pressed and deformed inward in the radial direction, an action portion 622c which abuts against and presses the proximal surface 621a of the pressing member 621, and a locking surface 622d which abuts against and locks the inner surface of the tube-shaped body 200 when being pressed and deformed inward in the radial direction (i.e., when the locking surface 622d contacts the inner surface of the tube-shaped body 200, further rotational movement of the plunger portion 622 is prevented).

The operation unit 622a is disposed in the opening portion 200c of the tube-shaped body 200. The operation unit 622a is an elastic material. The operation unit 622a is configured so that its original shape is recoverable after being pressed and deformed.

The fulcrum portion 622b is disposed on substantially the same plane of the outer surface of the tube-shaped body 200 (i.e., the fulcrum portion 622b is substantially planar with the outer surface of the tube-shaped body 200), and is integrally configured with the tube-shaped body 200. The fulcrum portion 622b may be separately configured as a member different from the tube-shaped body 200.

The action portion 622c protrudes substantially perpendicular to the operation unit 622a. When the operation unit 622a is pressed and deformed inward in the radial direction, the action portion 622c slides on the proximal surface 621a. When action portion 622c slides on the proximal surface 621a, the action portion 622c presses and moves the pressing member 621 to the distal side (i.e., distally).

The locking surface 622d on the proximal portion of the operation unit 622a extends in the axial direction of the lumen 200a of the tube-shaped body 200. When the operation unit 622a is pressed and deformed inward in the radial direction, the locking surface 622d abuts against and locks the inner surface of the tube-shaped body 200. In this manner, when being pressed and deformed, the proximal portion of the operation unit 622a is locked (i.e., further movement is prevented) by the locking surface 622d. Accordingly, the locking surface 622d serves as a starting point, and the operation unit 622a can recover its original shape.

Similarly to the first embodiment, the first elastic member 323 such as a coil spring and the like is disposed between the pressing member 621 and the engagement member 312.

The operation illustrated in FIGS. 12A to 12C is described below, in which the delivery mechanism 600 delivers the penetrating wire 11.

In an initial state before treatment using the penetrating wire 11 is performed, the outer periphery of the gripping member 311 is fitted to the engagement member 312 (as illustrated in FIG. 12A) in the delivery mechanism 600. The pinching piece 311a is restrained and is in a closed state (gripped state). In this state, the penetrating wire 11 is fixed to the gripping member 311 (i.e., the penetrating wire 11 and the gripping member 311 move together).

Subsequently, if a user (operator or the like) presses the plunger portion 622 inward in the radial direction, the action portion 622c contacts and slides on the proximal surface 621a. This contact presses/pushes the pressing member 621 to the distal side. As illustrated in FIG. 12B, the pressing member 621 moves distally and compresses the first elastic member 323. The gripping member 311 is interlocked with the pressing member 621 and so the gripping member 311 moves to the distal side. As illustrated in FIGS. 12A and 12B, the outer periphery of the gripping member 311 is fitted to the engagement member 312. The pinching piece 311a is restrained by the engagement member 312 and is thus in a closed state (gripped state). The penetrating wire 11 is fixed after being gripped by the gripping member 311. Accordingly, the penetrating wire 11 and the gripping member 311 move distally together by the same movement amount.

If the pressing member 621 is moved further to the distal side, the engagement member 312 abuts against the movement restriction member 331, and the movement of the engagement member 312 to the distal side is restricted. Accordingly, the gripping member 311 moves to the distal side further from the engagement member 312 (i.e., the gripping member 311 moves distally beyond the engagement member 312). Therefore, as illustrated in FIG. 12C, the gripping member 311 is released from the restraint of the engagement member 312, and loses the gripping force to grip the penetrating wire 11. In this manner, the penetrating wire 11 cannot be moved any further in the distal direction by the gripping member 311. The fixing portion 310 is thus in the unfixed position where the gripping member 311 releases the grip of the penetrating wire 11. In this position, similar to the first embodiment, the locking member 332b of the holding member 332 is fitted to the locking groove 332a, and thus, it is possible to hold the grip in the released position.

If the user releases the force to press the plunger portion 622 and the holding member 332 is no longer fitted, the operation unit 622a recovers its original shape due to the elastic force of the operation unit 622a included in the plunger portion 622. In addition, the pressing member 621 is pressed and moved to the proximal side by the first elastic member 323. In this manner, the gripping member 311 interlocked with the pressing member 621 moves to the proximal side, and returns to the initial state illustrated in FIG. 12A.

According to the above-described operations, the delivery mechanism 600 can perform the delivery operation for moving the penetrating wire 11 to the distal side by a predetermined movement amount. This operation is repeatedly performed, thereby enabling the delivered penetrating wire 11 to be delivered stepwise by a constant amount. Therefore, it is possible to suitably adjust the movement amount of the penetrating wire 11.

### Second Embodiment

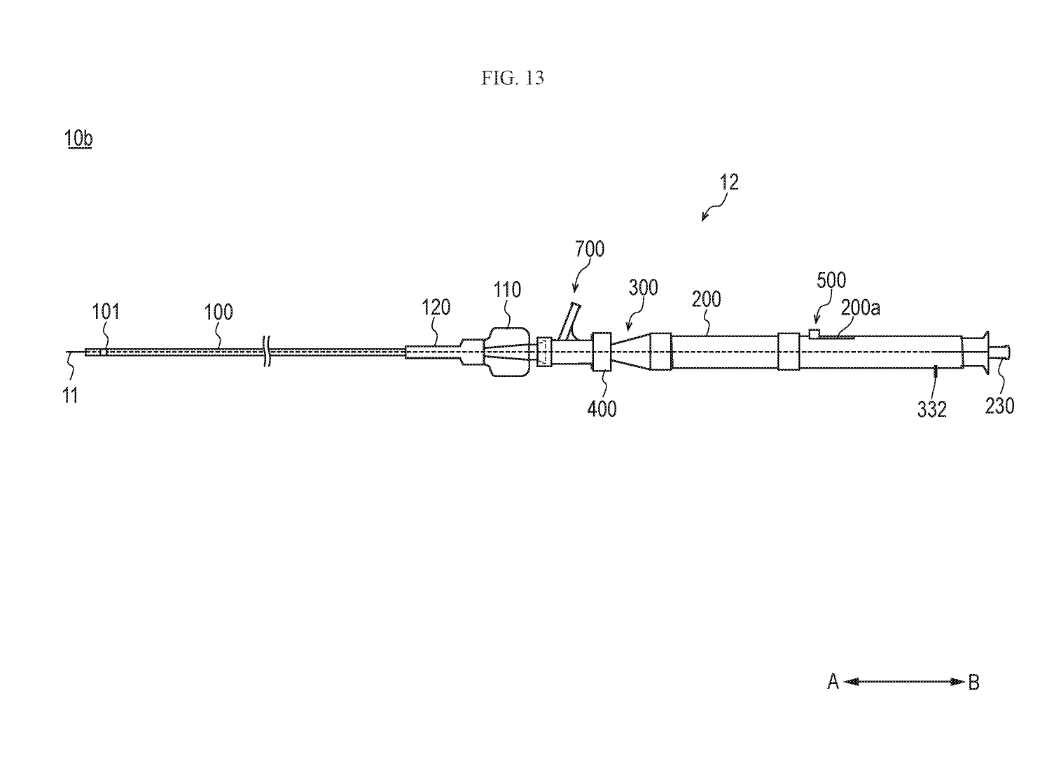

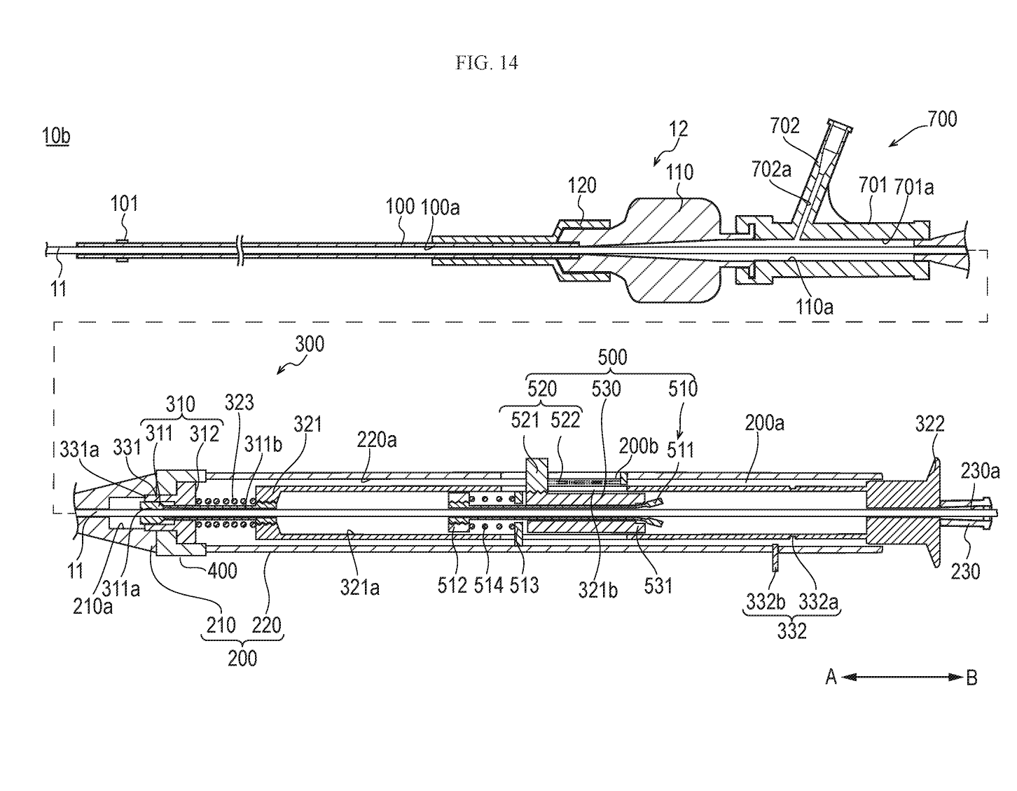

FIG. 13 is an overall configuration diagram of a medical device assembly 10b according to a second embodiment. FIG. 14 is a sectional view on a side extending along the longitudinal direction of the medical device assembly 10b according to the second embodiment. The same reference numerals are used for configurations which are the same as those according to the first embodiment, and thus, description of the same configurations will be omitted.

In brief, referring to FIGS. 13 and 14, the medical device assembly 10b according to the second embodiment further includes a connector 700 which interlocks the catheter 100 and the tube-shaped body 200 to be separable from each other. Configurations other than the connector 700 are substantially the same as those according to the first embodiment. For example, the connector 700 can be a Y-connector which is generally used for known catheter devices in the medical field.

The connector 700 has a main body portion 701 that communicates with the lumen 100a of the catheter 100. The main body portion 701 includes a first insertion path 701a into which the penetrating wire 11 is insertable. The connector 700 also has a bifurcated tube 702 that communicates with the first insertion path 701a. The bifurcated tube 702 has a second insertion path 702a through which a fluid circulates. The bifurcated tube 702 is bifurcated from the main body portion 701 as illustrated in FIGS. 13 and 14.

The catheter 100, the tube-shaped body 200, and the connector 700 are interlocked so as to be separable from each other. As a configuration for separable interlock, it is possible to use a known mechanical configuration such as fitting, screwing, and the like.

The tube-shaped body 200 may be configured so that the distal member 210 and the main body portion 220 which are included in the tube-shaped body 200 are interlocked to be separable from each other. Alternatively, the tube-shaped body 200 may be configured so that the distal member 210 and the main body portion 220 are not separable by being integrally fixed to each other.

The distal portion of the main body portion 701 is interlocked with the hub 110 connected to the proximal portion of the catheter 100. The distal portion of the main body portion 701 is separable from the hub 110. The proximal portion of the main body portion 701 is interlocked with the distal member 210 in the distal portion of the tube-shaped body 200 and is also separable from the distal member 210 of the tube-shaped body 200.

For example, the bifurcated tube 702 is interlocked via a predetermined fluid tube with a known fluid supply source (not illustrated) such as a prefilled syringe filled with a liquid of a drug, a contrast agent, or the like. Since the fluid supply source is interlocked with the bifurcated tube 702, a fluid, for example, such as a physiological salt solution, the contrast agent, a Ringer's solution, and the like can be supplied to the lumen 100a of the catheter 100 and the lumen 200a of the tube-shaped body 200 via the fluid tube and the second insertion path 702a of the bifurcated tube 702.

The connector 700 may be configured to include a valve body (not illustrated) between the main body portion 701 and the distal member 210. The valve body is disposed to freely open and close circulation of the fluid between the first insertion path 701a of the main body portion 701 and the insertion hole 210a of the distal member 210. A configuration material of the valve body is not particularly limited as long as the material has flexibility and liquid-tightness. It is possible to use elastic materials such as natural rubber, synthetic rubber, or various thermoplastic elastomers including polyamide-based elastomers, polyester-based elastomers for the valve body.

The material of the main body portion 701 and the bifurcated tube 702 is not particularly limited. For example, it is possible to use polyester such as polyvinyl chloride, polyethylene, polypropylene, cyclic polyolefin, polystyrene, poly-(4-methylpentene-1), polycarbonate, acrylic resin, acrylonitrile-butadiene-styrene copolymer, polyethylene terephthalate, polyethylene naphthalate, and the like, butadiene-styrene copolymer, polyamide (for example, nylon 6, nylon 6.6, nylon 6.10, nylon 12), or the like.

As described above, the medical device assembly 10b according to the second embodiment further has the connector 700 which interlocks the catheter 100 and the tube-shaped body 200 so as to be separable from each other. The connector 700 includes the main body portion 701 that communicates with the lumen 100a. that the main body portion 701 has the first insertion path 701a into which the penetrating wire 11 is inserted. The bifurcated tube 702 communicates with the first insertion path 701a and has the second insertion path 702a through which the fluid circulates. The bifurcated tube 702 and the second insertion path 702a bifurcate the main body portion 701 (i.e., the bifurcated tube 702 is located in the middle of the main body portion 701).

According to the medical device assembly 10b configured in this way, the catheter 100, the tube-shaped body 200 and the connector 700 can be interlocked so as to be separable from each other. Accordingly, for example, it is possible to use a combination of catheters 100 having different outer diameters or lengths and tube-shaped bodies 200 provided with different operation mechanisms. Therefore, it is possible to further improve usability.

### Modification Example 1 of Fixing Portion

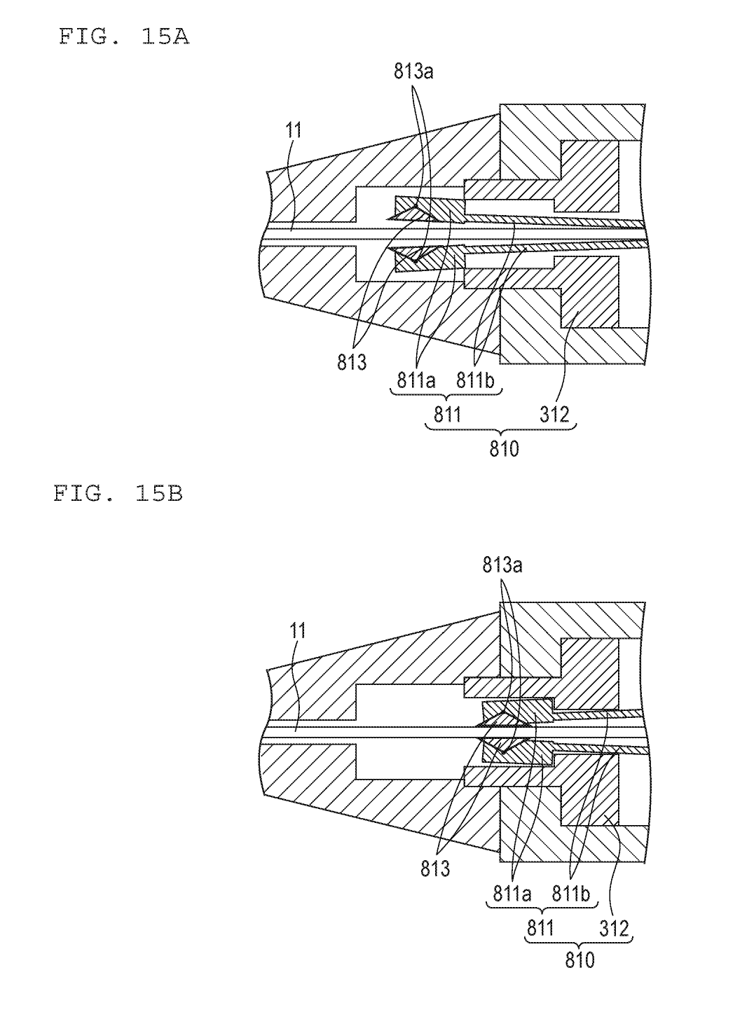

FIGS. 15A and 15B are views illustrating a gripping operation of a gripping member 811 according to Modification Example 1 of a fixing portion 810.

Similar to the first embodiment, the fixing portion 810 according to Modification Example 1 includes the gripping member 811 which grips the penetrating wire 11 and the engagement member 312 which is disposed at the outer periphery of the gripping member 811. The engagement member 312 has the same configuration as that of the first embodiment, and thus, description regarding the engagement member 312 is omitted.

The gripping member 811 has a pinching piece 811a which pinches the penetrating wire 11, and a support portion 811b which is connected to the proximal end of the pinching piece 811a. The support portion 811b is fixed to the moving unit 320.

When the pinching piece 811a is opened, the penetrating wire 11 gripped (fixed) by the gripping member 811 is released as illustrated in FIG. 15A. On the other hand, when the pinching piece 811a is closed as illustrated in FIG. 15B, the penetrating wire 11 is fixed after being gripped by the gripping member 811.

The pinching piece 811a has a hinge member 813 (corresponding to an adjusting portion 813) which adjusts a plane direction of a portion which is in contact with the penetrating wire 11 so as to be parallel to the extending direction of the penetrating wire 11, when the penetrating wire 11 is gripped (i.e., the contact portion of the pinching piece 811a is adjusted by the hinge member 813 to be parallel to the extending direction of the penetrating wire 11).

When the pinching piece 811a is in the open state (i.e., is not gripping the penetrating wire 11), as illustrated in FIG. 15A, the hinge member 813 is configured to follow the penetrating wire 11 to be inserted into each hinge member 813 and to be rotatable around a fulcrum 813a within a predetermined angular range. Accordingly, it is possible to improve insertion ability of the penetrating wire 11.

In addition, as illustrated in FIG. 15B, if the pinching piece 811a is pressed by the engagement member 312 from the outside, the pinching piece 811a is brought into a closed state (i.e., fixing/gripping the penetrating wire). At this time, the pinching piece 811 a tilts in the extending direction of the penetrating wire 11. The hinge member 813 pivots so that the contact surfaces in contact with the penetrating wire 11 are parallel to each other (i.e., the contact portion/surface of the pinching piece 811a is parallel to the extending direction of the penetrating wire 11). As a result, when the gripping member 811 grips the penetrating wire 11, the contact surface of the hinge member 813 is parallel to the extending direction of the penetrating wire 11 (i.e., the longitudinal direction).

As described above, the gripping member 811 according to Modification Example 1 of the fixing portion has the hinge member 813 which adjusts the plane direction of the portion which is in contact with the penetrating wire 11 so as to be parallel to the extending direction of the penetrating wire 11, when the penetrating wire 11 is fixed.

According to the fixing portion 810 configured in this way, when the gripping member 811 grips the penetrating wire 11 (regardless of the outer diameter of the penetrating wire 11), the penetrating wire 11 can be gripped by adjusting the contact surface of the pinching piece 811 a to be parallel to the extending direction of the penetrating wire 11. Therefore, various medical wires suitable for treatment conditions, such as the penetrating wire 11, can be selectively used.

### Modification Example 2 of Fixing Portion

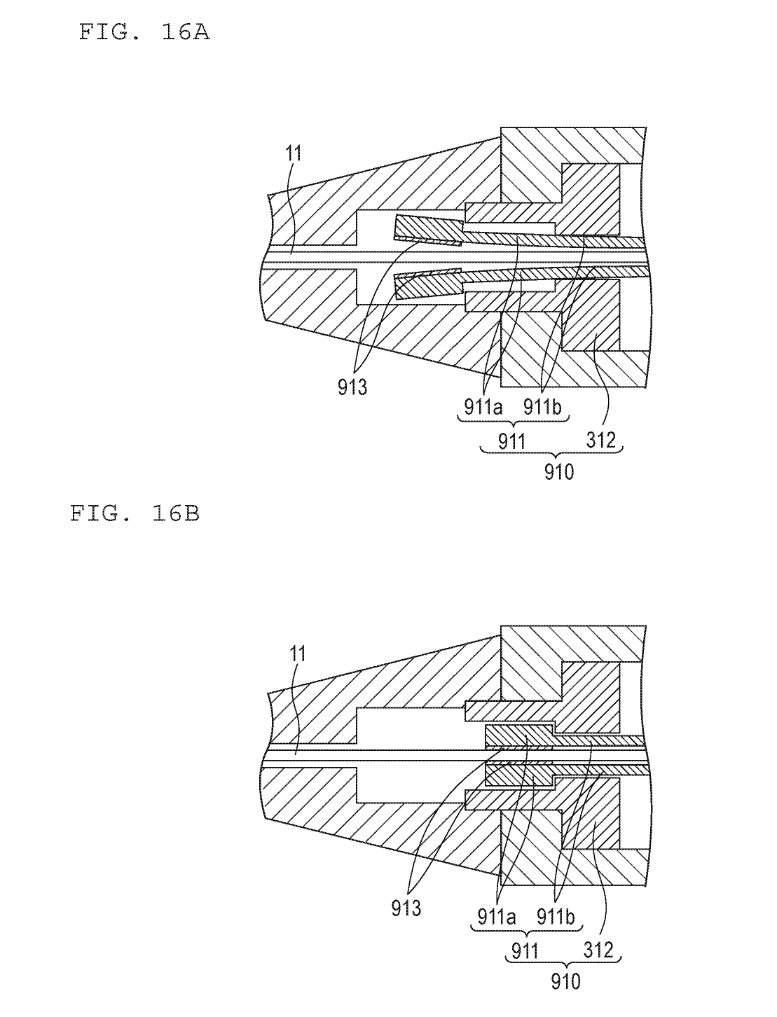

FIGS. 16A and 16B are views for describing a gripping operation of a gripping member 911 according to Modification Example 2 of the fixing portion.

Similar to the first embodiment, a fixing portion 910 according to Modification Example 2 has the gripping member 911 which grips the penetrating wire 11 and the engagement member 312 at the outer periphery of the gripping member 911. The engagement member 312 has the same configuration as that described in the first embodiment, and thus, a further description of the engagement member 312 is omitted.

The gripping member 911 has a pinching piece 911a which pinches the penetrating wire 11 and a support portion 911b. The support portion 911b is connected to the proximal end of the pinching piece 911a and is also fixed to the moving unit 320.

When the pinching piece 911a is opened, the penetrating wire 11 gripped (fixed) by the gripping member 911 is released as illustrated in FIG. 16A. On the other hand, as illustrated in FIG. 16B, when the pinching piece 911a is closed, the penetrating wire 11 is fixed after being gripped by the gripping member 911.

The pinching piece 911a has a flexible portion 913 obtained in such a way that a portion which is in contact with the penetrating wire 11 when the penetrating wire 11 is fixed is formed to be more flexible than other portions (i.e., the contact portion of the pinching piece 911a that contacts/grips the penetrating wire 11 is more flexible than other portions of the fixing portion 910).

The flexible portion 913 is formed of a flexible material. For example, as the flexible material, it is possible to use silicone, rubber, thermoplastic elastomers, butyl rubber, polyvinyl chloride, polybutadiene, polyurethane, or the like. Since the contact portion of the gripping member 911 is flexible, the gripping member 911 can grip the penetrating wire 11 to be aligned with the shape or the outer diameter of the penetrating wire 11. Therefore, the gripping force can be improved by increasing an area of the contact surface.

As described above, the gripping member 911 according to Modification Example 2 of the fixing portion has the flexible portion 913 obtained in such a way that the portion which is in contact with the penetrating wire 11 when the penetrating wire 11 is fixed is formed to be more flexible than other portions.

According to the fixing portion 910 configured in this way, it is possible to grip the penetrating wire 11 so as to be aligned with the shape or the outer diameter of the penetrating wire 11. Therefore, the gripping force can be improved by increasing the area of the contact surface.

The medical device and the medical device assembly have been described above with reference to the embodiments. However, without being limited to the configurations described in the embodiments, the medical device and the medical device assembly can be appropriately modified based on the description in appended claims.

For example, a configuration of the delivery mechanism and the delivery return mechanism is not limited to the configuration described in the embodiments, as long as the configuration is adopted in which the penetrating wire is moved to the distal side or the proximal side by a predetermined movement amount.

In addition, a configuration may include an adjusting portion which can adjust the movement amount enabling the delivery mechanism and/or the delivery return mechanism to move the penetrating wire by performing a single operation. For example, the adjusting portion adjusts the movement amount of the penetrating wire by adjusting extension and contraction lengths in the axial direction of the first elastic member included in the delivery mechanism and/or the second elastic member included in the delivery return mechanism. The proximal portion of the penetrating wire is provided with the marker. Therefore, the adjusted movement amount can be confirmed on the operator's hand operation side.

In addition, the delivery mechanism of the medical device assembly according to the second embodiment may have a configuration including the plunger portion pressed from the side surface portion as in the delivery mechanism of the modification example of the first embodiment.

In addition, the gripping member included in the fixing portion includes the adjusting portion or the flexible portion. However, the configuration may include both the adjusting portion and the flexible portion.

In addition, the medical device has the holding member that holds the fixing portion in the unfixed position (i.e., the penetrating wire is movable relative to the fixing portion). However, the configuration to unfix the fixing portion is not limited to the holding member. For example, a configuration may also be adopted in which the unfixed state is held by providing the first elastic member with a function to hold deformation.

In the above-described embodiments, the catheter device for delivering the medical wire (penetrating wire, guide wire) or penetrating the stenosed site have been described as examples. However, usage of the catheter is not particularly limited. For example, the disclosure here is applicable to various catheters such as a guiding catheter, an angiographic catheter, various balloon catheters for PTCA, PTA, IABP, and the like, an ultrasound catheter, an atherectomy catheter, an endoscopic catheter, an indwelling catheter, a drug solution administering catheter, or a micro catheter (embolectomy catheter) used in order to administer or inject various therapeutic drugs, embolic materials, or contrast agents to a target side inside organs (for example, liver) such as the brain and the abdomen. In particular, the catheter disclosed here may advantageously possess a decreased outer diameter. The catheter having the decreased diameter can effectively provide the above-described advantageous effects. Accordingly, the catheter may be a micro catheter and be configured to serve as a penetrating device. Therefore, it is possible to provide the catheter device which has the decreased diameter and which can perform effective treatment (penetrating the stenosed site).

The detailed description above describes embodiments of a medical device, medical device assembly and operational method representing examples of the inventive medical device, medical device assembly and operation disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A medical device comprising:
a catheter comprising a first lumen into which a medical wire is insertable, the catheter possessing a distal end and a proximal end;
a tube-shaped body comprising a second lumen configured to communicate with the first lumen, the tube-shaped body possessing a distal end, and the distal end of the tube-shaped body being proximal of the proximal end of the catheter; and
a delivery mechanism in the second lumen of the tube-shaped body, the delivery mechanism enabling a delivery operation to move the medical wire beyond the distal end of the catheter by a predetermined movement amount.

2. The medical device according to claim 1, wherein the delivery mechanism comprises:
a fixing portion configured to fix the medical wire so that the medical wire moves with the fixing portion;
a moving unit configured to move the fixing portion distally within the second lumen by the predetermined movement amount while the fixing portion fixes the medical wire; and
an unfixing portion configured to unfix the medical wire after the medical wire has been fixed by the fixing portion so that the medical wire is movable relative to the fixing portion.

3. The medical device according to claim 2, wherein
the fixing portion comprises a gripping member configured to grip the medical wire, the gripping member being expandable in the radial direction,
the fixing portion further comprises an engagement member which engages the gripping member to inhibit the gripping member from spreading outward in a radial direction, the engagement member holding the gripping member in the fixed position where the gripping member grips the medical wire when the engagement member engages the gripping member,
the moving unit comprises a pressing member configured to push and move the gripping member distally within the second lumen, and
the unfixing portion comprises a movement restriction portion which causes the gripping member and the engagement member to disengage from each other by restricting movement of the engagement member distally when the gripping member moves distally within the second lumen by more than the predetermined movement amount.

4. The medical device according to claim 2, wherein the fixing portion comprises a contact portion that contacts the medical wire to grip the medical wire when the medical wire is fixed, the contact portion being adjustable so that a plane direction of the contact portion is parallel to the longitudinal direction.

5. The medical device according to claim 2, wherein the fixing portion comprises a contact portion that contacts the medical wire to grip the medical wire when the medical wire is fixed, the contact portion being more flexible than other portions of the fixing portion.

6. The medical device according to claim 1, further comprising a rotary operation unit configured to rotate the medical wire around a rotation axis of the longitudinal axis.

7. The medical device according to claim 1, further comprising:
a delivery return mechanism configured to move the medical wire proximally; and
the delivery return mechanism comprising:
a delivery return fixing portion configured to fix the medical wire so that the medical wire moves with the delivery return fixing portion,
a delivery return moving unit configured to move the delivery return fixing portion proximally within the second lumen by a predetermined movement amount while the delivery return fixing portion fixes the medical wire, and
a delivery return unfixing portion configured to unfix the medical wire after the medical wire has been fixed by the delivery return fixing portion so that the medical wire is movable relative to the delivery return fixing portion.

8. The medical device according to claim 1, wherein
the delivery mechanism comprises a gripping member and a pressing member within the second lumen,
the gripping member is configured to grip the medical wire, and
the pressing member is operable to push the gripping member towards the distal end of the catheter while the gripping member grips the medical wire to move the medical wire by the predetermined movement amount.

9. The medical device according to claim 8, wherein the gripping member is configured to automatically release the medical wire when the pressing member moves the predetermined movement amount.

10. The medical device according to claim 8, wherein
the delivery mechanism comprises a plunger connected to the pressing member, the plunger extending proximally beyond the second lumen, the plunger possessing an outer diameter larger than an outer diameter of the tube-shaped body, and the plunger being movable relative to the tube-shaped body in the longitudinal direction to push the pressing member towards the distal end of the catheter.

11. A medical device assembly comprising:
an elongated medical wire configured to penetrate a biological lumen;
a medical device comprising:
a catheter including a first lumen,
an elongated tube-shaped body proximal to the catheter and comprising a second lumen that communicates with the first lumen, the medical wire being insertable into the first lumen via the second lumen, and
a delivery mechanism within the second lumen of the tube-shaped body configured to move the medical wire distally by a predetermined movement amount to penetrate the biological lumen; and the medical wire being assembled to the medical device such that the medical wire is attachable to and detachable from the medical device.

12. The medical device assembly according to claim 11, further comprising:
a connector that connects the catheter and the delivery mechanism to be separable from each other,
the connector comprising a main body portion with a first insertion path to communicate with the first lumen and into which the medical wire is inserted and a bifurcated portion with a second insertion path to communicate with the first insertion path to circulate a fluid, the bifurcated portion being bifurcated from the main body portion.

13. The medical device assembly according to claim 11, wherein the medical wire comprises a distal end possessing a distal shape to penetrate a stenosed site in the biological lumen.

14. The medical device assembly according to claim 11, wherein the medical wire comprises a scale to display a movement amount of the medical wire in an extending direction of the tube-shaped body.

15. The medical device assembly according to claim 11, wherein
the delivery mechanism comprises a gripping member and a pressing member within the second lumen,
the gripping member is configured to grip the medical wire, and
the pressing member is operable to push the gripping member towards the distal end of the catheter while the gripping member grips the medical wire to move the medical wire by the predetermined movement amount.

16. The medical device according to claim 15, wherein the gripping member is configured to automatically release the medical wire when the pressing member moves the predetermined movement amount.

17. The medical device according to claim 15, wherein
the delivery mechanism comprises a plunger connected to the pressing member, the plunger extending proximally beyond the second lumen, the plunger possessing an outer diameter larger than an outer diameter of the tube-shaped body, and the plunger being movable relative to the tube-shaped body in the longitudinal direction to push the pressing member towards the distal end of the catheter.

18. A method comprising:
inserting a medical device assembly into a living body, the medical device assembly comprising a lumen, a gripping member at least partially located in the lumen, and a penetrating wire within the lumen, the gripping member holding the penetrating wire in a fixed position so that the gripping member and the penetrating wire move together while the gripping member is gripping the penetrating wire;
moving the medical device assembly to an occluded stenosed site in the living body;
pushing the gripping member in a distal direction by a predetermined amount so that the penetrating wire moves distally by the predetermined amount to create a hole in the occluded stenosed site; and
the gripping member automatically releasing the penetrating wire when the gripping member has moved the predetermined amount such that further distal movement of the gripping member does not move the penetrating wire.

19. The method according to claim 18, further comprising
replacing the penetrating wire with a guide wire after the gripping member automatically releases the penetrating wire.

20. The method according to claim 19, wherein
the medical device assembly further comprises a second gripping member at least partially located in the lumen, the second gripping member holding the penetrating wire in a second fixed position so that the second gripping member and the penetrating wire move together while the second gripping member is gripping the penetrating wire, and the method further comprises:
retracting the second gripping member in a proximal direction by a second predetermined amount so that the penetrating wire moves proximally by the second predetermined amount after the gripping member has automatically released the penetrating wire.
